(19) United States
(12) Patent Application Publication
(10) Pub. No.: US 2015/0376216 A1
(43) Pub. Date: Dec. 31, 2015

(54) SUGAR-ANALOG PHOSPHORUS-CONTAINING HETEROCYCLES HAVING AN ANTI-METASTATIC ACTIVITY

(71) Applicants: CENTRE NATIONAL DE LA RECHERCHE SCIENTIRIQUE, Paris (FR); ECOLE NATIONALE SUPERIEURE DE CHIMIE DE MONTPELLIE, Montpellier (FR); UNIVERSITE MONTPELLIER 1, Montpellier (FR); UNIVERSITE MONTPELLIER 2 SCIENCES ET TECHNIQUES, Montpellier (FR); UNIVERSITE PARIS-NORD 13, Villetaneuse (FR); UNIVERSITE PARIS-SUD 11, Orsay (FR)

(72) Inventors: Norbert BAKALARA, SAINT MARTIN DE LONDRES (FR); Marcel DELAFORGE, EVRY (FR); Marc LECOUVEY, PARIS (FR); Jean-Philippe HUGNOT, MONTPELLIER (FR); Philippe LEGRAND, CASTELNAU LE NEZ (FR); Jean-Luc PIRAT, SAINT-AUNES (FR); David VIRIEUX, MONTPELLIER (FR); Jean-Noel VOLLE, JACOU (FR)

(21) Appl. No.: 14/769,928

(22) PCT Filed: Feb. 25, 2014
(86) PCT No.: PCT/FR2014/050409

§ 371 Date: Aug. 24, 2015

(30) Foreign Application Priority Data
Feb. 25, 2013 (FR) 1351654

(51) Int. Cl. C07F 9/6571; A61K 45/06; A61K 31/665

(52) U.S. Cl. CPC C07F 9/657172; A61K 31/665; A61K 45/06

(57) ABSTRACT

The use of compounds of formula (1) as defined in the description, for reducing or preventing the onset of metastases in a patient suffering from cancer. Pharmaceutical compositions for using in human or veterinary medicine, including at least one compound of formula (1) are also described.

### FIELD OF THE INVENTION

The present invention relates to the field of the therapeutic treatment of cancers in man or animals. The invention relates in particular to the use in human or veterinary medicine of a family of phosphorus-containing saccharide analogs (glycomimetics), incorporating a phosphinane ring, preferably oxaphosphinane, for its antimetastatic activity.

### PRIOR ART

According to recent statistics, cancer represents the second cause of mortality after cardiovascular diseases in industrialized countries. The means deployed for countering cancer-based diseases include strengthening early diagnosis, but also improving medicinal treatments. The discovery of novel original molecules, whose specificity toward tumor cells relative to healthy cells is total, would allow the development of new therapies.

In order to make a therapeutic choice for the treatment of a cancer, a certain number of parameters must be studied: the type of cancer (sarcomas, melanomas, etc.), the organ affected, the stage of evolution of the cancer, all the prognostic factors and the characteristics specific to the patient (age, general condition, mental state, etc.). From these data, a therapy may be chosen, which is either local or general. The most effective treatments are local therapies involving surgery and/or radiotherapy methods. They treat lesions that are not extensively developed and cure the majority of localized cancers. General therapies, chemotherapy and/or hormone therapy, are generally palliative or adjuvant treatments. These treatments are used in cases of localized but more extensively developed cancers. They make it possible to cure a limited number of generalized cancers, but improve the life expectancy of patients (cf. Capdeville R., Buchdunger E., Zimmermann J., Matter A., Nature Rev. Drug Discov., 2002, 1, 493; Eisenberg B. L., Von Mehren M., Expert Opin Pharmacother., 2003, 6, 869; Gilman A., Philips F. S., Science, 1946, 103, 409; Gingras D, Béliveau R. Med. Sci., 1997, 13, 1428-35.

The metastasis phenomenon is a complex series of steps during which cancer cells leave the original site (primary tumor) and migrate to other parties of the body. Cancer cells may in fact become detached from a primary tumor and, if the immune system does not detect them, may enter the lymphatic and blood vessels and then circulate to another part of the body to form a new tumor (“metastatize”). This is referred to as a secondary or metastatic tumor.

The formation of metastases is affected by many factors, and depends especially on the type of cancer and its aggressiveness. The risk of metastasis increases with the duration of presence of the primary tumor in the body, and it is its capacity for spreading to other tissues and organs that makes cancer a potentially mortal disease. The majority of people who die from a cancer have metastases at the time of their death, metastatic dissemination being the most frequent cause of death of cancer patients.

The most frequent cancers (for example prostate, breast, bowel and lung cancer) develop in organs that can be totally or partially removed by surgery. Surgery may cure the patients if the primary cancer has not metastatized. Most of the serious consequences of these cancers arise on account of their propagation to other parts of the body. In certain cases, the most serious effect of cancer is its propagation to a particularly essential part of the body. In other cases, propagation and growth in numerous organs creates so many cancer cells that the organism's normal metabolism is thereby profoundly disrupted.

The discovery of a primary tumor generally leads the physician to look for secondary foci. Despite the precision of the means available, the medical evaluation drawn up may remain negative, whereas months or years later, the appearance of visible metastases indicates that they were present but previously minimal and “hidden”. A cancer is generally only detectable once it reaches a certain volume: it is rare to be able to detect it before it reaches 1 cm in diameter, which corresponds to about 1 billion cells.

The treatment of a cancer therefore does not amount to treating the primary tumor (local antiproliferative treatment), but must quite often be accompanied by a preventive antimetastatic treatment, in particular when the risk of the cancer metastatizing is high, so as to hope for complete and prolonged remission. Specifically, during chemotherapy treatments, the action of the antiproliferative active principle brings out cells with a high migratory potential. This cell response to the treatment concerns a cellular subpopulation (of stem cell type) which has a molecular plasticity that affords it a response adapted to environmental variations. The aim of antimetastatic treatment is to block this adaptive response of evasion of the treatment via migration.

In certain cases, the primary tumor is not detected, either because it remained very small and invisible, or because it underwent a spontaneous regression after having released the malignant cells responsible for the metastatic foci.

The limitation concerning the use of the known anticancer agents concerns their high toxicity, which is the cause of a large number of side effects possibly leading as far as the death of the patient. The chemical weapons used for treating cancer are supposed to destroy the cancer cells while sparing the healthy cells. However, the selectivity is quite relative, and most of the medicaments used in chemotherapy have appreciable hematological toxicity. Reducing the harmful side effects, especially those with serious medical and psychological consequences, is just as important as attempting to improve the efficacy of a given medicament. On the whole, the current chemotherapeutic arsenal is still composed of old, highly cytotoxic medicaments, the majority of the known anticancer agents already being several decades old, poorly targeted, at the very least in cellular terms, and not affording any alternative to resistance phenomena. There is thus a need for novel anticancer molecules, which can be used in chemotherapy, and which, ideally, target only the cancer cells, and have antimetastatic properties.

Sugars, which represent a family of biomolecules that are ubiquitous in the living world, with a wide variety of structures and functions, have numerous applications in therapy: combating obesity and diabetes, but also as antiviral, antibiotic and anticancer agents. The synthesis of sugar analogs or glycomimetics is interesting, since these compounds are capable of interfering with the various receptors or enzymes involving sugars; in particular, the biosynthetic or energy mechanisms of certain molecules, glycoconjugates, and the mechanisms of adhesion between cells.

In the field of preparing cyclic saccharide analogs, two major approaches have already been developed: replacement of the hydroxyl group in the anomeric position with a carbon-based group (C-arylglycosides), and replacement of the endocyclic oxygen atom with another heteroatom (phosphosugars, phosphasugars, iminosugars, thioglycosides).

Recently, it has been shown in patent application WO 2009/004096 and in the article Clarion L. et al., J Med. Chem. 2012, 2196-2211, that a family of phosphinosugars has anticancer properties. The structure of a number of representative compounds of this family is indicated in the scheme below:

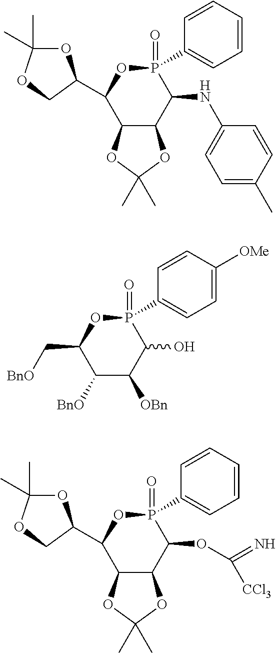

The compounds of this family show antiproliferative activity (cytotoxicity at low dose) toward several cancer cell lines, without showing cytotoxicity toward healthy (nontumoral) cells at the doses at which they have anticancer activity.

Surprisingly, the present inventors have now discovered that these heterocyclic phosphine compounds, which are sugar analogs, also have antimetastatic properties. The term “antimetastatic properties” means the property of reducing or preventing the appearance or propagation of metastases in a patient afflicted with a cancer. This may be reflected by an elimination of the formation of metastases or by a reduction in their size and/or the number of metastasis sites relative to the state that would be found in the absence of treatment.

For the purposes of the invention, the antiproliferative treatment of a secondary or metastatic cancer tumor (for example causing established metastases to regress or inhibiting the transformation of hidden metastases into macrometastases) is not considered as being an antimetastatic treatment involving the antimetastatic properties of the compounds according to the invention.

The compounds according to the invention thus target not only the multiplication of cancer cells by impairing the growth of the primary tumor or of metastatic tumors (antiproliferative curative action), but also target their mobility and inhibit or at the very least thus reduce the formation of metastases (preventive action).

Without wishing to be bound by a theory, the inventors think that the compounds according to the invention inhibit or reduce the molecular process of development of metastases and thus prevent or reduce the spread of metastases from primary tumors.

### SUMMARY OF THE INVENTION

The subject of the invention is the compounds of formula (1) below, referred to as phosphinosugars in the context of the present invention, for reducing or preventing the appearance of metastases in a patient afflicted with a cancer:

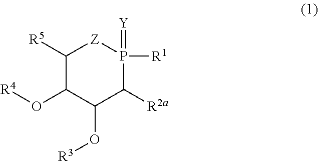

In this formula, Y represents an oxygen, sulfur or selenium atom, preferably an oxygen atom,

Z represents O, S, Se, NH or a group NR⁶, in which R⁶ is an optionally substituted aryl or alkyl group, preferably an oxygen atom,

R¹ represents a hydrogen atom, an optionally substituted alkyl group, or an aryl group,

R^{2a} represents a hydrogen or halogen atom, especially a fluorine atom, an azide group (N₃), a carbonate or dithiocarbonate group, a 1H-[1,2,3]triazolyl group or a group —X—R², X representing an oxygen, sulfur or selenium atom, a group NH or NR⁷, in which R⁷ is an optionally substituted aryl or alkyl group, X preferably representing O or NH, and R² representing an optionally substituted aryl or alkyl group, a hydrogen atom, a trichloroacetimidate group (—C(═NH)CCl₃), acyl, formyl, sulfonyl, sulfinyl, tert-butyldiphenylsilyl, allyl, a saccharyl, ester, amide, thioamide or sulfonamide group, or X—R² represents a group P(O)R^{2′}R^{6′} in which R^{2′} and R^{6′} denote, independently of each other, an optionally substituted aryl or alkyl group, OH, alkoxy or aryloxy,

R³ and R⁴ represent, independently of each other, an optionally substituted aryl or alkyl group, a hydrogen atom, a trichloroacetimidate, acyl, formyl, sulfonyl, sulfinyl, tert-butyldiphenylsilyl, allyl, ester, amide, thioamide, sulfonamide or saccharyl group or alternatively R³ and R⁴, taken together, form a divalent radical of formula —R³—R⁴—, in which —R³—R⁴— preferably represents an isopropylidene, benzylidene, diphenylmethylidene, cyclohexylmethylidene group, and substituted analogs thereof, for example a 4-methoxybenzylidene group, or a linear alkylene group such as the ethylene group (so as to form a propane-1,2-diol group),

R⁵ represents a hydrogen atom or a hydrocarbon-based group comprising one or more heteroatoms preferably chosen from oxygen, sulfur and nitrogen, better still oxygen.

The invention also provides anticancer pharmaceutical compositions for use in human or veterinary medicine, which comprise at least one compound of formula (1) in combination with one or more pharmaceutically acceptable excipients and/or vehicles, for reducing or preventing the appearance of metastases in a patient afflicted with a cancer.

The invention also relates to a method for the therapeutic treatment of man or animals, for reducing or preventing the appearance of metastases in an individual requiring such a treatment, said method comprising a step in which a therapeutically effective dose of a compound of formula (1) as defined in the present description is administered to the person or the animal, either alone or as a mixture with one or more pharmaceutically acceptable excipients and/or vehicles.

### DESCRIPTION OF THE FIGURES

FIG. 1 shows a measurement of the size of tumors presented by mice which have undergone a subcutaneous implantation of cancer cells of the SNB75 line (5×10⁶ cells) as a function of time and of various treatments. The mean standard deviation is also represented for each measurement.

FIG. 1A and FIG. 4 show photographs of mouse organs (liver or ovaries) which have or have not developed metastases depending on whether or not they have received a treatment with a compound according to the invention (3.48a or 4.2a), after they have received an injection of cancer cells.

FIGS. 2 and 3 represent diagrams illustrating the migration of cancer cells of the SNB75 and Gli4F11 lines, in a Boyden chamber, on various extracellular matrix protein supports, as a function of the concentration of compound 3.48a according to the invention.

FIG. 5 shows the mean number of metastases developed by mice after they have received an injection of cancer cells, depending on whether or not they have been treated with compound 4.2a according to the invention.

FIG. 6 represents the mass distribution of these secondary tumors as a function of the treatment conditions.

### DETAILED DESCRIPTION OF THE INVENTION

A family of phosphinosugar compounds comprising a 6-membered ring incorporating a phosphorus atom (phosphinane ring), typically a family of 1,2-oxaphosphinane 2-oxides, is provided according to the invention, in particular for its antimetastatic activity.

In the description of the chemical compounds, the terms are generally employed with their usual meaning.

In the present patent application, the term “alkyl” means a linear or branched, saturated or unsaturated hydrocarbon-based radical, containing from 1 to 25 carbon atoms, especially including acyclic groups containing from 1 to 8 carbon atoms such as methyl, ethyl, n-propyl, isopropyl, butyl and n-hexyl groups, the cycloalkyl groups preferably containing from 3 to 7 carbon atoms, the cycloalkylmethyl groups preferably containing from 4 to 8 carbon atoms.

The term “substituted alkyl” group means an alkyl group as defined above, connected via an sp^{a} carbon atom and substituted with one or more aryl groups and/or comprising one or more heteroatoms such as N, S or O. Examples that will be mentioned include arylalkyl groups such as the trityl group (—CPh₃), the benzyl group noted as Bn or the 4-methoxybenzyl group, alkoxyalkyl groups, especially dialkoxymethyl groups such as diethoxymethyl or dimethoxymethyl groups, groups CH₂CO₂R¹¹, in which R¹¹ represents an optionally substituted alkyl or aryl group.

The term “alkoxy” denotes an alkyl group connected to the rest of the molecule via an oxygen atom, for example an ethoxy, methoxy or n-propoxy group.

The term “aryloxy” denotes an aryl group connected to the rest of the molecule via an oxygen atom, for example a benzoxy group.

The term “acyl” denotes a radical derived from a carboxylic acid by deletion of the hydroxyl group, preferably having the formula —C(O)R⁸, R⁸ denoting an optionally substituted aryl or alkyl group, for example acetyl, propionyl, oleoyl, myristoyl, benzoyl or trifluoroacetyl groups.

The term “sulfonyl” denotes a radical derived from a sulfonic acid by deletion of the hydroxyl group, preferably having the formula —SO₂R⁹, R⁹ denoting an optionally substituted aryl or alkyl group, for example a CF₃ group.

The term “sulfinyl” denotes a radical derived from a sulfinic acid by deletion of the hydroxyl group, preferably having the formula —SOR¹⁰, R¹⁰ denoting an optionally substituted aryl or alkyl group.

The term “dithiocarbonate” denotes a group of formula —OC(S)SR^{9c}, R^{9c} denoting an optionally substituted aryl or alkyl group.

The term “carbonate” denotes a group of formula —OC(O)OR^{9d}, R^{9d} denoting an optionally substituted aryl or alkyl group.

The term “ester group” denotes a group of formula —C(O)OR^{10′}, R^{10′} denoting an optionally substituted aryl or alkyl group.

The term “amide group” denotes a group of formula —C(O)NR^{9′}R^{9″}, R^{9′} denoting an optionally substituted aryl or alkyl group and R^{9″} denoting an optionally substituted aryl or alkyl group or a hydrogen atom, for example a group —C(O)NHPh.

The term “thioamide group” denotes a group of formula —C(S)NR^{9a}R^{9b}, R^{9a} denoting an optionally substituted aryl or alkyl group and R^{9b} denoting an optionally substituted aryl or alkyl group or a hydrogen atom.

The term “sulfonamide group” denotes a group of formula —SO₂NR^{11′}R^{11″}, R^{11′} denoting an optionally substituted aryl or alkyl group and R^{11″} denoting an optionally substituted aryl or alkyl group or a hydrogen atom.

The term “aryl” denotes an aromatic monovalent carbocyclic radical comprising only one ring (for example a phenyl group) or several fused rings (for example naphthyl or terphenyl groups), which may optionally be substituted with one or more groups such as, without limitation, alkyl (for example methyl), hydroxyalkyl, aminoalkyl, hydroxyl, thiol, amino, halo (fluoro, bromo, iodo or chloro), nitro, alkylthio, alkoxy (for example methoxy), aryloxy, monoalkylamino, dialkylamino, acyl, carboxyl, alkoxycarbonyl, aryloxycarbonyl, hydroxysulfonyl, alkoxysulfonyl, aryloxysulfonyl, alkylsulfonyl, alkylsulfinyl, cyano, trifluoromethyl, tetrazolyl, carbamoyl, alkylcarbamoyl or dialkylcarbamoyl groups. Alternatively, two adjacent positions of the aromatic ring may be substituted with a methylenedioxy or ethylenedioxy group.

The term “aryl” also includes “heteroaryl” groups, i.e. aromatic rings in which one or more carbon atoms of the aromatic ring(s) are substituted with a heteroatom such as nitrogen, oxygen, phosphorus or sulfur. The heteroaryl groups may be structures containing one or more aromatic rings, or structures containing one or more aromatic rings coupled with one or more nonaromatic rings. In the structures bearing several rings, the rings may be fused, covalently linked or linked via a common divalent group such as a methylene, ethylene or carbonyl group. Examples of heteroaryl groups are thiophene (2-thienyl, 3-thienyl), pyridine (2-pyridyl, 3-pyridyl, 4-pyridyl), isoxazole, phthalimide, pyrazole, indole, furan groups and benzo-fused analogs thereof, phenyl pyridyl ketone, quinoline, phenothiazine, carbazole, benzopyranone.

The term “saccharyl group” as used herein covers all radicals derived by deletion of a hydroxyl group or a hydrogen atom (preferably a hydroxyl group) from a natural or synthetic, protected or unprotected carbohydrate or sugar. The saccharyl group includes monosaccharyl and oligosaccharyl groups such as disaccharyl groups. The saccharyl groups, for example glucosyl or mannosyl groups, may be derived from sugars such as, without limitation, glucuronic acid, lactose, sucrose, maltose, allose, alltrose, glucose, mannose, idose, galactose, talose, ribose, arabinose, xylose, lyxose, fructose, threose, erythrose, β-D-N-acetylgalactosamine, β-D-N-acetylglucosamine, fucose, sialic acid, N-acetylneuraminic acid, N-acetylmuramic acid, glucosamine, galactosamine, rhamnose and analogs thereof that are protected or substituted, for example with acyl, alkyl, aryl, halo or amino groups, and also deoxy analogs thereof. The term “oligosaccharyl group” means a saccharyl group derived from at least two covalently linked monosaccharides, preferably comprising from 1 to 3 saccharide units. The preferred saccharyl groups are monosaccharyl groups. In the compounds of formula (1), when R^{2a} represents —X—R², R² being a saccharyl group, this saccharyl group is preferably linked via a group X representing 0 or NH, preferably 0. For a description of structures of saccharide type, reference may be made to the book “Essentials of Glycobiology”, Varki et al. Eds., chapter 2 (Cold Spring Harbor Press, Cold Spring Harbor, N.Y., 1999).

The term “saccharide” means a monosaccharide or an oligosaccharide. Bn represents the benzyl group, Ac the acetyl group.

Certain compounds of the invention may exist in both solvated and nonsolvated form, for example in hydrate form. In general, the solvated forms are equivalent to the nonsolvated forms and are included in the scope of the present invention. Certain compounds of the present invention may exist in multiple crystalline or amorphous forms. In general, all the physical forms are equivalent for the uses envisaged by the present invention and are included in the scope of the present invention.

The compounds according to the invention bear several asymmetric (optical) centers, and as such enantiomers or diasteroisomers may exist. It is understood that the invention covers all the enantiomers and diasteroisomers of the compounds of formula (1) and mixtures thereof, especially the racemates. In other words, the compounds according to the invention may be used in the form of a purified enantiomer or in the form of a mixture of enantiomers. The various isomers may be separated according to methods known to those skilled in the art, especially by chromatography on silica gel or by fractional crystallization.

The preferred compounds of formula (1) are those in which Y=Z=O, i.e. 1,2-oxaphosphinane 2-oxides.

In the compounds according to the invention, the substituent R¹, when it does not denote a hydrogen atom, is always connected to the endocyclic phosphorus atom via a carbon atom.

The preferred groups R¹ are the groups H, alkyl, such as the 2-benzyloxyethyl, ethyl, n-butyl, 3-phenylpropyl or n-octyl group, dialkoxymethyl such as diethoxymethyl or dimethoxymethyl groups, aryl, such as the phenyl, 4-methylphenyl, 4-nitrophenyl, 4-aminophenyl, 4-methoxyphenyl, 3,4-difluorophenyl, 3,5-difluorophenyl, 2-thienyl, 4-fluorophenyl, 4-biphenyl, 3-methylphenyl or 3-methoxyphenyl groups and also the following groups:

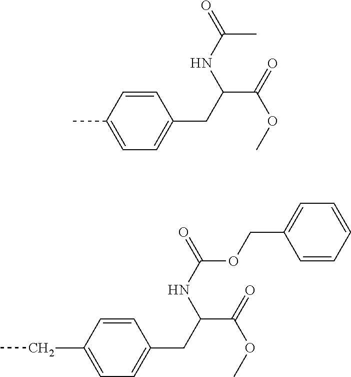

The preferred groups R² are the groups H, arylsulfonyl, methylsulfonyl, trichloroacetimidate, benzyl, saccharyl, and aryl, such as phenyl, 4-methylphenyl, 4-nitrophenyl, 4-aminophenyl, 3,4-difluorophenyl, 3,5-difluorophenyl and 3,4-dinitrophenyl.

The preferred groups X—R² are the groups O-aryl, OH, NH₂, NH-aryl, S-aryl, dithiocarbonate, NHCH₂CO₂R¹¹, R¹¹ having the meaning indicated above, NHC(O)R¹², R¹² representing an optionally substituted alkyl or aryl group, O—SO₂R⁹, R⁹ having the meaning indicated above, NH-Bn, O-saccharyl, OC(═NH)CCl₃, phosphonic acid, phosphinic acid or phosphine oxide, urea, thiourea, carbamate or carbonate.

Preferably, R³ and R⁴ represent, independently of each other, a hydrogen atom, a benzyl, benzoyl or acetyl group, or together form a divalent radical of formula —R³—R⁴— preferably representing an isopropylidene group.

According to a preferred embodiment of the invention, the group R⁵ is such that the compounds (1) correspond to formula (2) or (3):

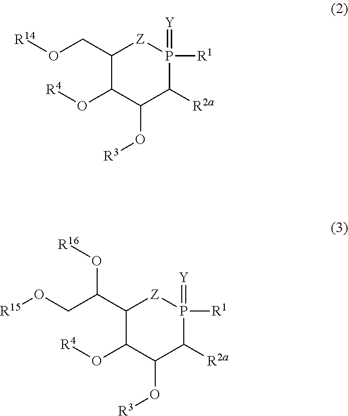

in which R¹, R^{2a}, R³, Y and Z have the same meanings as previously, R¹⁴, R¹⁵ and R¹⁶ represent, independently of each other, a hydrogen atom, an optionally substituted aryl or alkyl group, a trichloroacetimidate, acyl, formyl, sulfonyl, sulfinyl, tert-butyldiphenylsilyl, allyl, ester, amide, thioamide, sulfonamide or saccharyl group or alternatively R¹⁵ and R¹⁶, taken together, form a divalent radical of formula —R¹⁵—R¹⁶—, in which —R¹⁵—R¹⁶— preferably represents an isopropylidene, benzylidene, diphenylmethylidene or cyclohexylmethylidene group, and substituted analogs thereof, for example a 4-methoxybenzylidene group, or a linear alkylene group such as an ethylene group.

The group R⁵, when it does not represent a hydrogen atom, preferably comprises from 1 to 25 carbon atoms, preferably from 1 to 20 carbon atoms, better still from 1 to 10 carbon atoms and even better still from 1 to 8 carbon atoms. The group R⁵ may be an optionally substituted alkyl group comprising one or more heteroatoms preferably chosen from oxygen, sulfur and nitrogen, better still oxygen. The preferred groups R⁵ are alkoxyalkyl groups such as the benzyloxymethyl group (—CH₂OBn), —CH₂OH, 2,2-dimethyl[1,3]dioxolan-4-yl and 1,2-dihydroxyethyl CH(OH)CH₂OH, which is reflected, in formulae (2) and (3), by R¹⁴=H or Bn, and R¹⁵=R¹⁶=H or R¹⁵ and R¹⁶, taken together, form an isopropylidene radical.

The various methods for preparing the compounds of formula (1) have been described in detail in patent application WO 2009/004096, and the article Clarion L. et al. J Med. Chem. 2012, 2196-2211, which are incorporated herein by reference.

Examples of preferred families of compound (1) are those having the following general formulae, in which R¹ and R^{2a} are as defined previously:

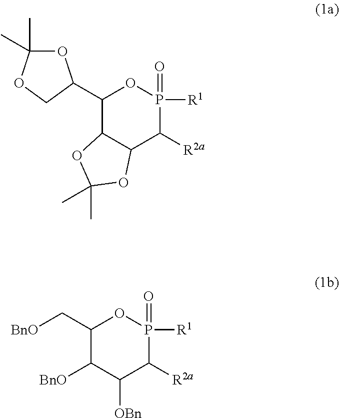

Among the 1,2-oxaphosphinanes of general formula (18), the compounds of formulae (19) and (20) are preferred, in which R¹ and R³ to R⁵ are as defined previously, R¹ preferably representing an aryl group, a hydrogen atom, an optionally substituted alkyl group such as a dialkoxymethyl group:

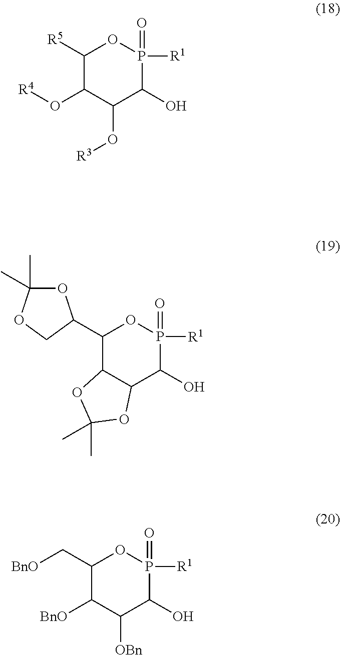

A second preferred class of compounds (1) according to the invention corresponds to the 1,2-oxaphosphinanes of general formula (21):

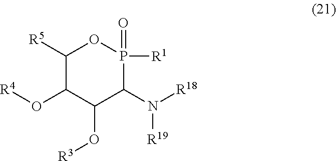

in which R¹ and R³ to R⁵ have the same meaning as previously, R¹⁸ and R¹⁹ represent, independently of each other, a hydrogen atom, an aryl group, an optionally substituted alkyl group, a trichloroacetimidate, acyl, formyl, sulfonyl, sulfinyl, tert-butyldiphenylsilyl, allyl, ester, amide, thioamide or sulfonamide group, R¹⁸ preferably representing a hydrogen atom. Among these nitrogenous phosphinosugars, mention may be made of those comprising an amino group in position C₃ (R¹⁸=R¹⁹=H), a benzylamino group in position C₃ (R¹⁸=H, R¹⁹=Bn) or an acetylamino group in position C₃ (R¹⁸=H, R¹⁹=Ac). Among the compounds (21), the compounds of formulae (22) and (23) are preferred, R¹ and R¹⁹ having the meaning indicated previously, R¹ preferably representing an aryl group, a hydrogen atom or a dialkoxymethyl group and R¹⁹ preferably representing an aryl or acyl group:

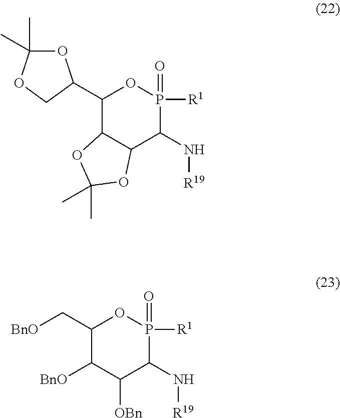

A third class of preferred compounds (1) corresponds to the following compounds of formula (24), in which the various substituents have the meanings indicated previously and R²⁰ denotes a saccharyl group, preferably monosaccharyl.

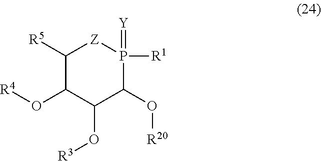

Among the compounds of formula (24), the preferred compounds are the pseudo-disaccharides of formulae (27), (27a) and (27b) below (derived from coupling with a mannose or glucose derivative), in which R^{19a}, R^{19b}, R^{19c}, R^{19d} R^{19e}, R^{19f} and R^{19g} denote, independently of each other, a hydrogen atom or a benzyl group, R^{19″} denotes a hydrogen atom or a methyl group, the other substituents being as defined previously:

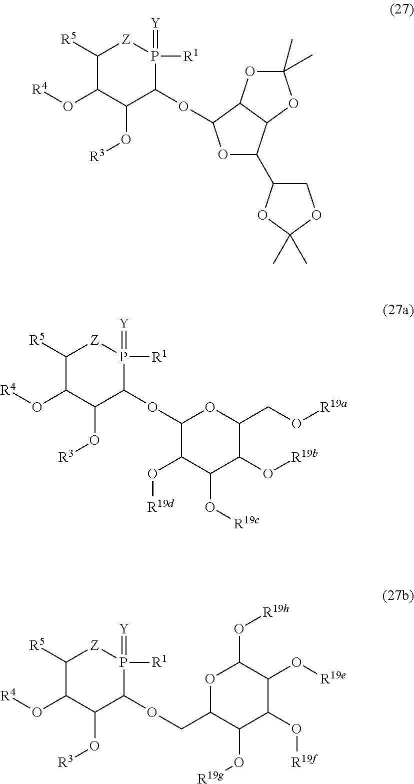

Another preferred class of compounds (1) corresponds to the following compounds of formula (24a), in which the various substituents having the meanings indicated previously. Among these compounds, those of formulae (24b) and (24c) are preferred:

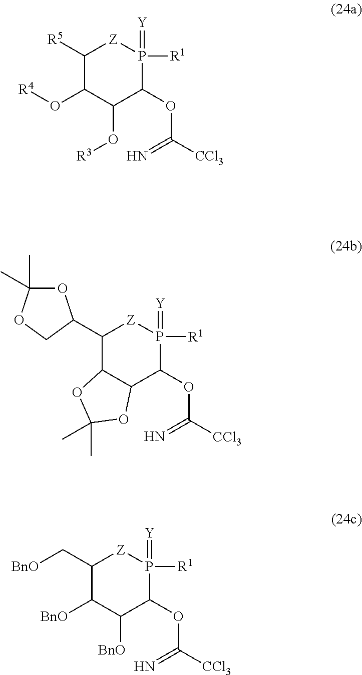

The compounds (1) in which R¹ is a hydrogen atom, noted as compounds (28), and the compounds (30) in which R²¹ represents an aryl group, are also preferred compounds, the aryl groups possibly representing groups as diverse as phenyl, 2,4-difluorophenyl, 3,4-difluorophenyl, 4-methoxyphenyl, 3,4-dinitrophenyl or 4-nitrophenyl groups:

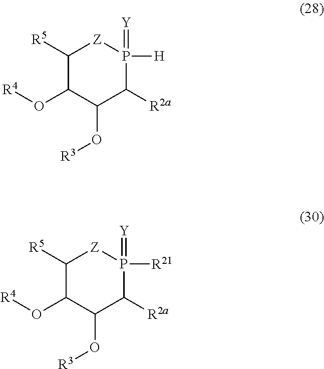

Although the phosphinosugars (1) according to the invention may be obtained in a form in which their hydroxyl groups are protected, the invention is in no way limited to this embodiment and also covers the phosphinosugars (1) obtained in a form in which all their hydroxyl groups are deprotected, or only some of them. The preferred polyhydroxy phosphinosugars corresponding to this other embodiment of the invention, and also other preferred phosphinosugars, correspond to the formulae below, in which R¹, R^{2a}, Y and Z have the meaning indicated previously, R²² denotes a group CF₃ or an aryl group, for example the 4-tolyl group (R^{2a} preferably representing a group OSO₂R²² as defined above, a group N₃ or a halogen atom, preferably a fluorine atom), and R³⁰ and R³¹ denote, independently of each other, a hydrocarbon-based group or a hydrogen atom:

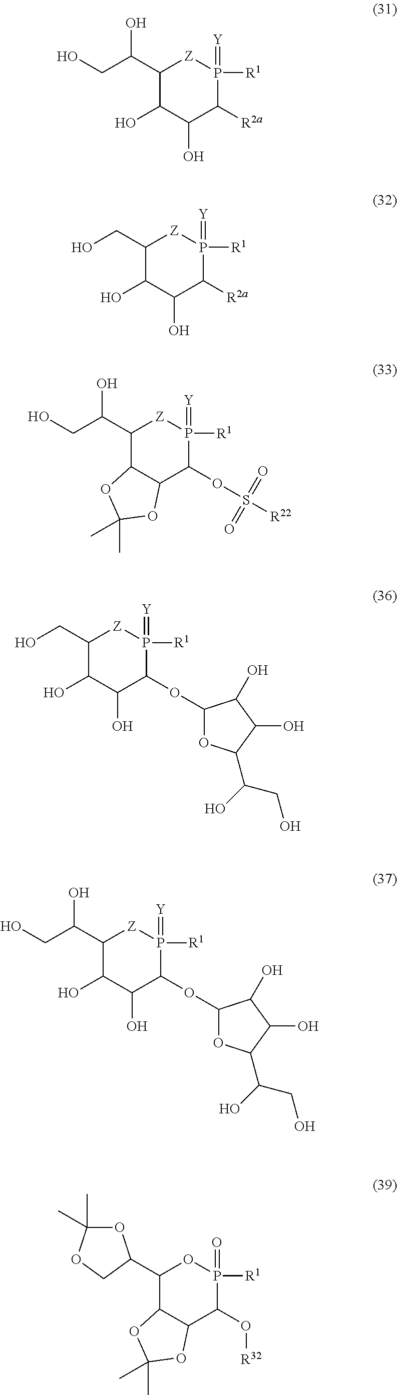

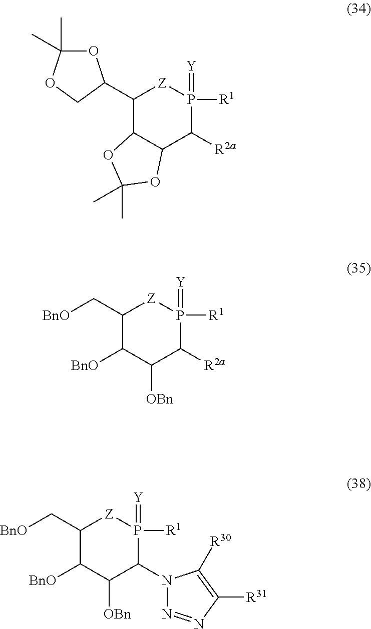

In formula (39), R¹ has the meaning indicated previously and R³² is chosen from the same substituents as those indicated previously for the group R¹⁹. R³² preferably represents an acyl group, preferably the benzoyl group. In formula (38), R³⁰ and R³¹ preferably represent, independently of each other, an alkyl or aryl group or a hydrogen atom. Preference will be given to the compounds of formula (38) in which one of the groups R³⁰ and R³¹ is a hydrogen atom, and the other in a hydrocarbon-based group, better still those in which R³⁰=H and R³¹=aryl, ideally phenyl. The compounds of formula (38), comprising a 1H[1,2,3]triazolyl substituent, may especially be obtained by reacting the corresponding azides with an alkyne (for example by azide-alkyne cycloaddition catalyzed with copper(I)). The preferred compound of formula (38) has the following structure (38a):

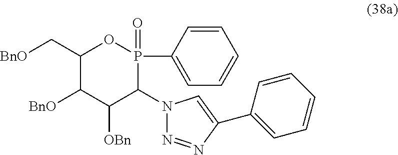 [SMILES: O=P1(c2ccccc2)OC(COCc2ccccc2)C(OCc2ccccc2)C(OCc2ccccc2)C1n1cc(-c2ccccc2)nn1]

One of the preferred compounds of formula (35) has the following structure (35a):

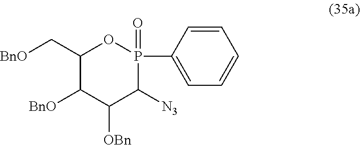 [SMILES: [N-]=[N+]=NC1C(OCc2ccccc2)C(OCc2ccccc2)C(COCc2ccccc2)OP1(=O)c1ccccc1]

The compounds of formula (1) in which R²² is a halogen atom (preferably fluorine, chlorine or bromine) may be obtained from the corresponding triflate (R^{2a}=OSO₂CF₃, which is itself obtained from the compound in which R^{2a}=OH), by nucleophilic substitution in the presence of a suitable halide salt. Use may be made especially of the ammonium salts n-Bu₄N⁺X⁻ (X=F, Cl, Br, I). It is also possible to directly convert the compounds of formula (1) in which R^{2a}=OH into corresponding haloderivatives by reaction with Castro's reagent (Ph₃PX₂ with X=Cl, Br, I) or Appel's reagent (Ph₃P, CX₄ with X=Cl, Br, I).

The following classes of compound of formula (1) are preferred: those bearing an aromatic nucleus on the phosphorus atom, and/or the mannose derivatives bearing a heteroatom substituted in position C1 α to the phosphorus atom, and/or those bearing protection of the hydroxyl groups in the non-anomeric position.

According to one embodiment, the following compounds are excluded from the products and/or methods of the invention:

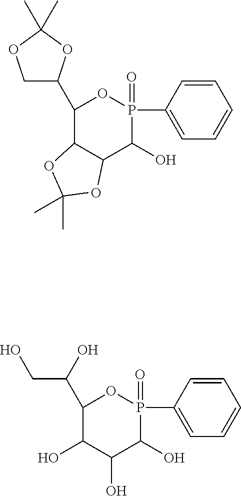 [SMILES: CC1(C)OCC(C2OP(=O)(c3ccccc3)C(O)C3OC(C)(C)OC23)O1 ; O=P1(c2ccccc2)OC(C(O)CO)C(O)C(O)C1O]

### Compositions Comprising a Compound of Formula (1) According to the Invention

As has already been mentioned previously in the present description, the compounds of formula (1) have, besides antiproliferative anticancer activity, antimetastatic activity in the sense that they inhibit or reduce the migration of cancer cells, and thus constitute active principles that may be used in any type of anticancer composition.

The invention relates to a compound of formula (1) as defined in the present description, for its use as a medicament for human or veterinary use, for reducing or preventing the appearance of metastases in a patient afflicted by a cancer.

In particular, the compounds of formula (1) are useful as active principles in pharmaceutical compositions for human or veterinary use, intended for treating cancers (metastatic or primary), i.e. cancer cells, or for preventing the appearance of cancers (for example the compounds of formula (38)), especially for reducing or preventing the appearance of metastases in a patient afflicted by a cancer. In the case where the patient is afflicted by a metastatic cancer, the compounds of formula (1) are especially directed in particular toward reducing or preventing the appearance of additional metastases.

In the present description, a patient denotes both an animal, in particular a nonhuman mammal, and a person. The term “patient afflicted by a cancer” means both a patient afflicted by a declared cancer (primary or metastatic) and a hidden cancer, i.e. invisible, the existence of which has been revealed, for example, by the discovery of metastases.

A subject of the invention is also a pharmaceutical composition for use in human or veterinary medicine comprising at least one compound of formula (1) as defined in the present description, preferably in combination with one or more pharmaceutically acceptable excipients and/or vehicles, in particular for reducing or preventing the appearance of metastases in a patient afflicted by a cancer.

In the present patent application, cancer cells denote cells having typical characteristics of cells that cause cancer, such as uncontrolled proliferation, immortality, metastatic potential, rapid growth and high speed of proliferation, and certain specific morphological characteristics. Cancer cells are often in the form of a tumor, but such cells may exist alone in the body, or may be non-tumor-forming cancer cells, such as leukemic cells. Cancer cells may be associated with numerous types of cancers, comprising, without limitation, leukemia, a lymphoma, a melanoma, a neuroblastoma, liver cancer, ovarian cancer, brain cancer, lung cancer, bowel cancer, breast cancer, pancreatic cancer, prostate cancer, testicular cancer, esophageal cancer, uterine cancer, cervical cancer, kidney cancer, stomach cancer, bladder cancer, a cerebrospinal cancer or a colorectal cancer. The pharmaceutical compositions of the invention may be used for the therapeutic treatment of at least one of the cancers mentioned above.

When the compounds according to the invention are used in the context of an antimetastatic treatment, the patient is afflicted with a “primary” cancer. This cancer is a cancer that is capable of metastatizing, which may be, without limitation, a melanoma, a glioblastoma multiforme, a lung cancer, especially non-small-cell lung cancer, bowel cancer or colorectal cancer, breast cancer, prostate cancer, testicular cancer, cervical cancer, kidney cancer, preferably a glioblastoma multiforme, breast cancer or non-small-cell lung cancer. The compounds of the invention are particularly suited for treating the risk of metastasis in a patient afflicted with a glioblastoma multiforme. It is now recognized that glioblastoma multiforme (GBM), commonly known as glioblastoma, may be a cancer with metastatic potential giving rise to a generalized pathology (Schönsteiner, S. S. et al., Journal of Clinical Oncology 2011, 29, 23, 668-671). Cancer cells originating from glioblastomas may effectively cross the blood-brain barrier and establish extraneural metastates. The reported sites of extraneural metastases are the lungs, the pleura, the liver, cervical lymphatic nodules, bones and bone marrow.

A human or veterinary pharmaceutical composition according to the invention may also comprise one or more other active principles different from a compound of formula (1), especially for increasing its efficacy, including one or more other anticancer compounds (antiproliferative and/or antimetastatic). As other active principles that may be included in a pharmaceutical composition according to the invention, mention may be made especially of antihistamine agents, antiinflammatory agents, disinfectants or local anesthetics.

According to one embodiment of the invention, the composition according to the invention contains, as anticancer active principles, only compounds of formula (1).

According to another embodiment of the invention, the composition according to the invention contains an additional anticancer agent, such as a taxane (for example paclitaxel or docetaxel), vinblastine, vinorelbine, vincristine, bleomycin, temozolomide, 5-fluorouracil and/or an angiogenesis inhibitor (for example bevacizumab).

Alternatively or additionally, the composition according to the invention may be used in combination with another therapeutic treatment, especially in combination with an additional anticancer treatment administered separately, such as a treatment using one of the additional anticancer agents mentioned above, for example targeting the proliferation of cancer cells and/or angiogenesis.

Thus, the invention also relates to a combination product for simultaneous or separate use or for use spread out over time, for the treatment of a cancer, comprising at least one cytotoxic compound (intended for treating proliferation of said cancer), and at least one compound of formula (1) according to the invention for reducing or preventing the appearance of metastases.

A pharmaceutical composition comprising an anticancer compound according to the invention is, without preference, in a solid form (dry particles) or in a liquid form. In the liquid form, preference will be given to a pharmaceutical composition in the form of an aqueous suspension or a nonaqueous suspension, or alternatively in the form of a water-in-oil or oil-in-water emulsion.

Among the pharmaceutical compositions according to the invention, mention may be made more particularly of those that are suitable for oral, topical, parenteral, nasal, intravenous, percutaneous (transcutaneous), subcutaneous, rectal, perlingual or respiratory administration and especially simple or sugar-coated tablets, sublingual tablets, gel capsules, lozenges, suppositories, creams, ointments, dermal gels and drinkable or injectable vials.

The dosage varies according to the sex, age and weight of the patient, according to the administration route, and according to the type of cancer, the state of progress of the cancer, in particular according to whether or not metastases have been detected in the patient. The dosage may also vary according to the type of combined anticancer treatment(s).

In general, a compound of formula (1) as defined in the present description is used in amounts preferably ranging from 0.001 mg/kg of body weight of the patient or of the animal to 1 g/kg of body weight of the patient or of the animal per 24 hours, in one or more dosage intakes. Preferably, said amount is at least equal to 0.01 mg/kg and better still 0.05 mg/kg.

Preferably, said amount is not more than 500 mg/kg and better still 100 mg/kg.

For an oral administration, a pharmaceutical composition according to the invention may be in the form of tablets, gel capsules, wafer capsules, sugar-coated tablets, syrups, suspensions, solutions, powders, granules, emulsions, suspensions of microspheres or of nanospheres, suspensions of lipid vesicles or of vesicles based on various polymers.

For an oral administration, a pharmaceutical composition according to the invention may be in the form of tablets that may be manufactured from solid compositions comprising at least one compound of formula (1) in combination with various excipients such as microcrystalline cellulose, sodium citrate, calcium carbonate, dicalcium phosphate or glycine. Use may be made of various disintegrants such as starch (corn, potato, tapioca, etc. starch), alginic acid or a silicate. Use may be made of binders such as polyvinylpyrrolidone, sucrose, gelatin or acacia. Use may be made of lubricants such as magnesium stearate, sodium lauryl sulfate or talc. Such solid compositions, in the form of a powder, may be used for the manufacture of gelatin capsules. For solid compositions, use may also be made of lactose or a high molecular weight polyethylene glycol.

To manufacture liquid compositions for oral administration, the compound of formula (1) may be combined with various sweeteners, flavorings, colorants, optionally also with emulsifiers or suspension agents, in combination with diluents such as water, ethanol, a propylene glycol, glycerol or any combination of these excipients.

For a parenteral administration, a pharmaceutical composition according to the invention may in the form of solutions or suspensions for perfusion or injection.

For a parenteral administration, use may be made in particular of oily or aqueous solutions or alternatively of suspensions, emulsions or implants including suppositories. For example, a compound of formula (1) may be dispersed in a liquid vehicle such as a physiological saline liquid or alternatively a saline solution containing 5% by weight of dextrose, which are conventionally used for the preparation of injectable pharmaceutical formulations.

For an enteral administration, use may be made of controlled-release compositions, for example compositions in which the compound of formula (1) is protected from the external medium by a plurality of coating layers that degrade differently, for example on contact with a neutral or basic medium (enteric coating layers) or in contact with an aqueous medium (coating layers comprising soluble polymers or polymers which degrade in water).

The pharmaceutical composition of the present invention may be used for parenteral, topical or local administration and prophylactically and/or therapeutically. Thus, the anticancer compound according to the present invention is prepared in a form suited to the chosen type of administration, for example in liquid form or in lyophilized form.

The pharmaceutical compositions comprising an anticancer compound according to the invention may contain a liquid or solid, for example aqueous, pharmaceutically acceptable excipient and/or vehicle. Many pharmaceutically acceptable excipients and/or vehicles may be used, for example solvents or diluents; water, where appropriate as a mixture with propylene glycol or polyethylene glycol, buffered water, a saline solution, a solution of glycine and derivatives thereof, a nonaqueous solution especially comprising solvents such as ethanol, N-methylpyrrolidone, dimethylacetamide (DMA), dimethyl sulfoxide (DMSO) and/or dimethylformamide (DMF), and also agents required for reproducing the physiological conditions, for instance buffers and pH adjusters, surfactants such as Solutol® HS15, Tween® 80, sodium acetate, sodium lactate, sodium chloride, potassium chloride, calcium chloride, or a vehicle such as Cremophor EL®, this list not being limiting. Furthermore, the pharmaceutical composition may be sterilized via sterilization techniques that are well known to those skilled in the art. It is preferred to use at least one organic solvent and at least one surfactant for dissolving the compounds of formula (1), preferably a mixture of ethanol, dimethylacetamide and at least one surfactant.

As inert, nontoxic pharmaceutically acceptable vehicles, adjuvants or excipients, mention may also be made, as nonlimiting guides, of solubilizers other than solvents, preserving agents, wetting agents, emulsifiers, dispersants, binders, swelling agents, disintegrants, encapsulating agents, retardants, lubricants, absorbers, suspension agents, colorants, flavorings, stabilizers, thickeners, etc. Such compounds are, for example, magnesium carbonate, magnesium stearate, talc, lactose, pectin, dextrin, starch, gelatin, cellulose-based materials, cocoa butter, etc.

In general, a pharmaceutical composition according to the invention comprises from 0.01% to 99% by weight and advantageously from 1% to 90% by weight of compounds of formula (1), relative to the total weight of said composition. The dose of compounds (1) administered daily generally ranges from 0.5 to 50 mg/kg, and preferably from 1 to 20 mg/kg. In general, a pharmaceutical composition according to the invention comprises from 1% to 99.99% by weight and advantageously from 10% to 99% by weight of a pharmaceutically acceptable excipient and/or vehicle (or diluent) or of a combination of pharmaceutically acceptable excipients and/or vehicles.

When a solid composition in tablet form is prepared, the main active ingredient is mixed with a pharmaceutical vehicle such as gelatin, starch, lactose, magnesium stearate, talc, gum arabic or the like.

The tablets may be coated with sucrose or other suitable starting materials or alternatively may be treated such that they have prolonged or delayed activity and so that they continuously release a predetermined amount of active principle.

A preparation as gel capsules is obtained by mixing the active ingredient with a diluent and pouring the mixture obtained into soft or hard gel capsules.

A pharmaceutical composition in syrup or elixir form may contain the active ingredient in combination with a sweetener, preferably a calorie-free sweetener, methylparaben and propylparaben as antiseptics, and also an agent giving taste and a suitable colorant.

The water-dispersible powders or granules may contain the active ingredient as a mixture with dispersants or wetting agents or suspension agents, for instance polyvinylpyrrolidone, and similarly with sweeteners or flavor enhancers.

The active principle may also be formulated in the form of microcapsules, optionally with one or more supports or additives.

In general, to manufacture a pharmaceutical composition in accordance with the invention, a person skilled in the art may advantageously refer to the latest edition of the European Pharmacopea, for example the 5th edition of the European Pharmacopea published in January 2005, or alternatively the 6th edition of the European Pharmacopea, available to the public in June 2007.

Techniques for preparing pharmaceutical compositions according to the invention may be readily found by a person skilled in the art, for example in the book Remmingston's Pharmaceutical Sciences, Mid. Publishing Co, Easton, Pa., USA.

Physiologically acceptable vehicles and excipients (or adjuvants) are also described in the book entitled “Handbook of Pharmaceutical Excipients”, Second edition, American Pharmaceutical Association, 1994.

To formulate a pharmaceutical composition according to the invention, a person skilled in the art may advantageously refer to the latest edition of the European Pharmacopea or of the American Pharmacopea (U.S. Pharmacopea, especially the edition USP 30-NF 25).

Advantageously, a pharmaceutical composition as defined above is adapted for oral, parenteral or intravenous administration.

When the pharmaceutical composition according to the invention comprises at least one pharmaceutically or physiologically acceptable excipient, it is in particular an excipient that is suitable for oral administration of the composition or an excipient that is suitable for parenteral administration of the composition.

The invention also relates to a therapeutic treatment method, for preventing or treating the development of a cancer in a patient, and/or for reducing or preventing the appearance of metastases in a patient afflicted by a cancer, said method comprising a step during which a therapeutically effective amount of a compound of formula (1) as defined in the present description or of a pharmaceutical composition containing said compound of formula (1) is administered to the patient.

The antimetastatic treatment according to the invention is generally an anticancer treatment that is complementary or adjuvant to a main treatment. This adjuvant treatment is generally applied in a second stage when, following the initial treatment, information prompting a therapeutic supplement is collected. However, in certain cases, this adjuvant treatment may be applied first, when the indications in favor of this complementary treatment are known from the outset. If the analyses performed substantiate the hypothesis of microscopic dissemination in the organism, a general treatment via chemotherapy using compounds according to the invention is justified for treating this generalization before metastases appear.

The invention may be performed in combination with other treatment modes, such as hormone therapy, surgery, cryotherapy, hyperthermia, radiotherapy, an additional chemotherapy, etc. Advantageously, the treatment according to the invention may be combined with a treatment targeting the proliferation of cancer cells and/or angiogenesis.

The invention is also illustrated, without, however, being thereby limited, by the examples that follow.

### Examples

### a) Materials

Unless otherwise indicated, the NMR spectra were recorded on a Bruker Avance 400 spectrometer operating at 400.13 MHz (¹H) in CDCl₃. The chemical shifts are expressed in ppm/TMS for ¹H and ¹³C; the coupling constants ⁿJ are expressed in Hz. When the spectra are first-order, or may be considered as such, the signals are denoted by the letters s (singlet), d (doublet), t (triplet), q (quartet), m (multiplet) and by combinations of these letters. A broad signal will be denoted by one of these letters, preceded by the letter b.

The high-resolution mass spectra (HR MS) were recorded on a Jeol JMS DX-300 machine in positive FAB ionization mode with p-nitrobenzyl alcohol (NBA) as matrix.

The moisture-sensitive or oxygen-sensitive compounds were handled under nitrogen using Schlenk techniques. The anhydrous solvents are distilled under nitrogen with the appropriate drying agent. Separation of the products by flash chromatography is performed on a column of Merck 15-40 μm or 30-75 μm silica gel.

Nude female mice 4-5 weeks old with an average weight of 25 mg, supplied by the R. Janvier breeding center, were used. The mice were kept in a pathogen-free environment and with food and water present ad libitum. The experiments were commenced after one week of acclimatizing the mice to their new environment.

Several cancer cell lines were used for the in vivo or in vitro biological tests:
SNB75 is a human glioblastoma line derived from a primary culture, i.e. derived from a primary malignant cancer tumor. It forms part of the panel of 59 cancer lines referenced by the NCI. Its Cosmic reference number published by the Welcome Trust Sanger Institute is 905982. This line was supplied by Mr Souhgheng Ning (M.D. Ph.D of the department of “Radiation Oncology”, Stanford University Medical Center). It was cultured in Ham's F-12 and DMEM (1/1) supplemented with 10% fetal bovine serum. The experiments were performed with cells in the exponential growth phase.
Gli4 (or Gli4F11) and Gli7 are cultures of cancer strain cells derived from human glioblastomas (primary cultures). These lines are CD133⁺, CD15⁺ which are multipotent and tumorigenic in animals. They were established by J. P. Hugnot (INM INSERM U-1051 Eq. 4), and described in Guichet, P. O. et al, Glia 2013, 61(2), 225-239;
C6 is a rat glioblastoma line. It is referenced in the ATCC under the reference CCL-107.
MDAMB-435 is a melanoma line derived from a mammary melanoma. It forms part of the panel of 59 cancer lines referenced by the NCI. It is referenced at the ATCC under the reference HTB-129. It was cultured in Ham's F-12 and DMEM (1/1) supplemented with 10% fetal bovine serum. The experiments were performed with cells in the exponential growth phase.

### b) Preparation of Compounds of Oxazaphosphinane Nature

### b.1) Compound 3.48a (4,5-bis-benzyloxy-6-benzyloxymethyl-2-phenyl-2-oxo-2λ⁵-[1,2]oxaphosphinan-3-ol)

500 mg (0.9 mmol) of the mixture of diastereomers of formula 3.47 comprising a P—H bond described in patent application WO 2009/004096, 125 mg of tetrakis(triphenylphosphine)palladium (0.09 mmol, 0.1 eq.), 5 ml of anhydrous toluene, iodobenzene or bromobenzene (0.9 mmol, 1 eq.) and 365 μl of triethylamine (2.7 mmol, 3 eq.) are successively introduced into a three-necked flask under nitrogen equipped with a condenser. The reaction medium is maintained at 70° C. for 4 hours with magnetic stirring. The reaction medium is filtered through Celite and the filtrate thus obtained is evaporated under reduced pressure. The oily residue is then purified by chromatography on silica gel (100/0 to 50/50 CH₂Cl₂/EtOAc gradient), making it possible to separate two diastereomers 3.48a and 3.48b obtained in an 81/19 mole ratio. The diastereoisomer 3.48a is obtained in the form of a colorless solid in an overall yield of 60% (C₃₂H₃₃O₆P, M=544.59 g/mol). A more efficient method for preparing this compound was described in the publication J. Med. Chem. 2012, 55, 2196-2211.

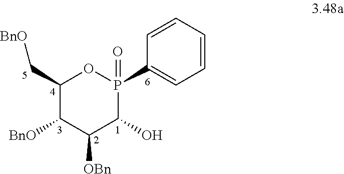 [SMILES: O=[P@@]1(c2ccccc2)O[C@H](COCc2ccccc2)[C@@H](OCc2ccccc2)[C@H](OCc2ccccc2)[C@H]1O]

¹H NMR (400.13 MHz, CDCl₃): δ 3.68 (dd, 1H, ³J_{H-H}=1.9 Hz, ²J_{H-H}=11.1 Hz, ⁵CH₂ₐ), 3.86-3.91 (m, 2H, ⁵CH_{2b}+⁴CH), 3.94 (bd, 1H, ²J_{P-H}=9.6 Hz, ¹CH), 4.11 (m, 1H, ³CH), 4.44-4.63 (m, 4H, 2 CH₂OBn), 4.80-4.84 (m, 2H, CH₂OBn), 4.89 (dd, 1H, ³J_{P-H}=11.3 Hz, ³J_{H-H}=4.7 Hz, ²CH), 7.12-7.74 (m, 20H, CH_{Ar}).

HR MS⁺ (NBA): Theoretical mass 545.2093; Experimental mass 545.2104.

### b.2 Compound 4.2a (S_{P}RSRR)-3-benzoate-4-(2,2-dimethyl-[1,3]dioxolan-4-yl)-2,2-dimethyl-2-oxo-2-phenyl-tetrahydro-2λ⁵-[1,2]oxaphosphinane)

Benzoic anhydride (132 mg, 0.58 mmol), pyridine (25 μl, 0.39 mmol) and DMAP (4.8 mg, 0.039 mmol) are added, under a nitrogen atmosphere, to a solution of compound 3.3a described in patent application WO 2009/004096 (150 mg, 0.39 mmol) in anhydrous CH₂Cl₂ (3 ml). The reaction mixture is stirred at room temperature overnight. The organic solution is washed with water and with saturated aqueous sodium chloride solution. The combined organic phases are dried over sodium sulfate, filtered and concentrated under vacuum. The residue is dissolved in the minimum required amount of CH₂Cl₂ and reprecipitated from hexane. Compound 4.2a is obtained in the form of a colorless solid in an overall yield of 78% (149 mg).

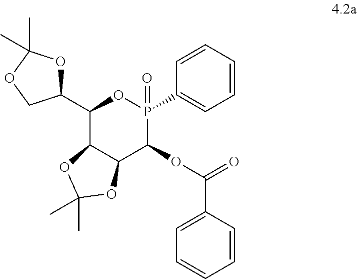 [SMILES: CC1(C)O[C@H]2[C@@H]([C@H]3COC(C)(C)O3)O[P@](=O)(c3ccccc3)[C@@H](OC(=O)c3ccccc3)[C@H]2O1]

³¹P NMR (161.97 MHz, DMSOd₆): δ=25.02 ppm. ¹H NMR (400.13 MHz, DMSO d₆): δ=1.30 (s, 3H), 1.33 (s, 3H), 1.38 (s, 3H), 1.56 (s, 3H), 3.86 (dd, 1H, J=5.8 Hz), 4.06 (dd, 1H, J=7.8 Hz), 4.31 (dd, 1H, J=12.6 Hz), 4.70 (d, 1H, J=8 Hz), 4.86 (d, 1H, J=6 Hz), 4.98 (ddd, 1H, J=24, 8, 4 Hz), 6.00 (d, 1H, J=4 Hz), 7.53-7.97 (m, 10H) ppm. ¹³C NMR (100.61 MHz, DMSOd₆): δ=24.39, 25.16, 25.62, 26.28, 65.48, 67.16 (d, J=105.3 Hz), 73.41 (d, J=8.05 Hz), 73.46 (d, J=6 Hz), 73.93 (d, J=8.05 Hz), 75.72 (d, J=8.05 Hz), 108.79, 109.92, 128.29, 128.68 (d, J=13.2 Hz), 129.18 (d, J=51.2 Hz), 130.78 (d, J=10.2 Hz), 131.28 (d, J=134.7 Hz), 132.73 (d, J=2.4 Hz), 134.04, 164.78 ppm. HRMS (ESI) m/z calculated for C₂₅H₃₀O₈P (M+H)⁺: 489.1678. found: 489.1669.

### b.3) Synthesis of 5,6-Di-O-isopropylidene-N-benzyl-D-mannosylamine

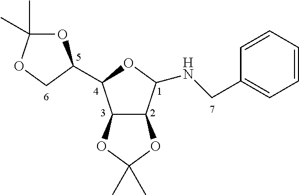

10 g of protected D-mannose ([2,3,5,6]-di-O-isopropylidene-D-mannose, described in patent application WO 2009/004096; 38 mmol), 100 ml of ethanol, 10 g of magnesium sulfate and 7.5 ml of distilled benzylamine (57 mmol, 1.5 eq.) are placed in a three-necked flask. The medium is stirred at reflux for 48 hours. The medium is filtered through Celite and the filtrate obtained is evaporated under reduced pressure. The oily residue is directly purified on a column of silica gel with a dichloromethane/ethyl acetate system in a gradient from 100/0 to 50/50. The product is obtained in the form of a yellow oil. Yld 58%.

¹H NMR (400.13 MHz, CDCl₃): δ 1.19 (s, 3H, CH₃), 1.24 (s, 3H, CH₃), 1.30 (s, 3H, CH₃), 1.38 (s, 3H, CH₃), 3.30 (dd, 1H, ³J_{H-H}=3.3 Hz, ³J_{H-H}=7.6 Hz, ⁴CH), 3.67 (dd, 1H, ³J_{H-H}=5.9 Hz, ³J_{H-H}=7.2 Hz, ⁵CH), 3.81 (dd, 1H, ²J_{H-H}=3.8 Hz, ³J_{H-H}=2.0 Hz, ²CH), 3.97 (m, 2H, ⁶CH₂), 4.29 (td, 1H, ³J_{H-H}=3.3 Hz, ³CH), 4.48 (ddd, 2H, ²J_{H-H}=41.8 Hz, ³J_{H-H}=6.3 Hz, ²J_{H-H}=3.5 Hz, ⁷CH₂), 4.66 (dd, 1H, ³J_{H-H}=5.1 Hz, ³J_{H-H}=3.6 Hz, ¹CH), 4.81 (s, 1H, NH), 7.22-7.47 (m, 5H, CH_{Ar}).

### Synthesis of (R_{P}SSRR)-4-(2,2-Dimethyl-[1,3]dioxolan-4-yl)-2,2-dimethyl-2-oxo-2-phenyl-tetrahydro-6λ*5*-[1,3]dioxolo[4,5-d][1,2]oxaphosphinan-3-aminobenzyl

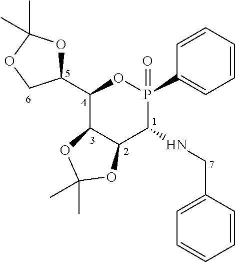 [SMILES: CC1(C)O[C@@H]2[C@H](O1)[C@@H](NCc1ccccc1)[P@](=O)(c1ccccc1)O[C@@H]2[C@H]1COC(C)(C)O1]

1 g of the compound obtained in the preceding paragraph (3 mmol), 10 ml of THF, 0.35 ml of methyl phenylphosphinate (3 mmol, 1 eq.) and potassium tert-butoxide (70 mg, 0.2 eq.) are placed in a Schlenk tube, deoxygenated beforehand and under a nitrogen atmosphere. The medium is thus stirred at room temperature for 15 hours. 15 ml of 0.1N hydrochloric acid are then added to the reaction medium, followed by 15 ml of saturated sodium chloride solution and 50 ml of chloroform. The aqueous phase is extracted with twice 50 ml of chloroform. The organic phases are combined, dried over magnesium sulfate and evaporated under reduced pressure. The oily residue thus obtained is then purified on a column of silica gel using diethyl ether. 73% yield.

³¹P NMR (161.97 MHz, CDCl₃): 538.31. ¹H NMR (400.13 MHz, CDCl₃): δ 1.35 (s, 3H, CH₃), 1.37 (s, 3H, CH₃), 1.40 (s, 3H, CH₃), 1.45 (s, 3H, CH₃), 3.35 (dd, 1H, ³J_{H-H}=4.3 Hz, ²J_{P-H}=7.6 Hz, ¹CH), 3.94 (bs, 2H, ⁷CH₂), 4.14 (m, 2H, ⁶CH₂), 4.43 (m, 1H, ³CH), 4.49 (qd, 1H, ³J_{H-H}=1.8 Hz, ³J_{H-H}=7.6 Hz, ⁴CH), 4.58 (m, 1H, ⁵CH), 4.69 (ddd, 1H, ³J_{P-H}=21.2 Hz, ³J_{H-H}=4.3 Hz, ³J_{H-H}=6.6 Hz, ²CH), 7.23-7.97 (m, 5H, CH_{Ar}). ¹³C NMR (100.61 MHz, CDCl₃): δ 24.66 (1s, 1C, CH₃), 25.26 (1s, 1C, CH₃), 26.31 (1s, 1C, CH₃), 27.01 (1s, 1C, CH₃), 52.97 (d, 1C, ¹J_{P-C}=87.3 Hz, ¹CH), 53.05 (s, 1C, ⁷CH2), 66.81 (s, 1C, ⁶CH₂), 72.56 (d, 1C, ²J_{P-C}=3.6 Hz, ²CH), 73.70 (d, 1C, ³J_{P-C}=5.1 Hz, ⁵CH), 73.81 (d, 1C, ³J_{P-C}=8.7 Hz, ³CH), 75.47 (d, 1C, ²J_{P-C}=1.5 Hz, ⁴CH), 109.10 (s, 1C, Cq), 110.01 (s, 1C, Cq), 127.27 (s, 2C, CH_{ANI} m), 128.14 (s, 2C, CH_{ANI} o), 128.79 (s, 1C, CH_{Ar} m), 128.30 (s, 1C, CH_{Ar} m), 129.57 (d, 1C, J_{P-C}=138.9 Hz, Cq_{Ar}), 129.60 (s, 1C, CH _{ANI} p), 131.82 (s, 1C, CH_{Ar} o), 131.97 (s, 1C, CH_{Ar} o), 132.87 (s, 1C, CH_{Ar} p), 138.94 (d, 1C, ³J_{P-C}=17.4 Hz, Cq_{Ar}). IR (KBr pellet): 3351 ff (v NH), 2980 ff, 2894 ff (v CH_{Alk}), 1491 ff, 1439 ff, 1370 f, 1265 f, 1205 FF, 1167 f, 1128 m (v C—O—C), 1059 f (v P═O), 962 m (v P—O—C), 925 m (5 CH_{Ar}), 891 m, 817 f, 733 F, 713 f, 693 F. MS FAB+ (NBA): m/z (%) 475 [MH+H⁺] (90%); 474 [M+H⁺] (50%). HR MS⁺ (NBA): Theoretical mass 474.2046; Experimental mass 474.2058.

### Synthesis of (R_{P}SSRR)-(2,2)-Dimethyl-[1,3]dioxolan-4-yl)-2,2-dimethyl-2-oxo-2-phenyl-tetrahydro-6λ*5*-[1,3]dioxolo[4,5-d][1,2]oxaphosphinin-3-amino 5.5d

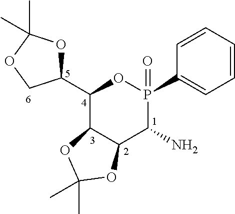 [SMILES: CC1(C)O[C@@H]2[C@H](O1)[C@@H](N)[P@](=O)(c1ccccc1)O[C@@H]2[C@H]1COC(C)(C)O1]

1 g of phosphinosugar (0.002 mol), 10 ml of ethanol, and 1 g of palladium-on-charcoal are placed in a Schlenk tube, deoxygenated beforehand, under a nitrogen atmosphere. The medium is thus stirred at room temperature for 24 hours under a hydrogen atmosphere. The reaction medium is then filtered through Celite to remove the palladium. The filtrate obtained is evaporated under reduced pressure to give a slightly colored oil. This oil is purified on a column of silica gel, the expected product only emerging on eluting with ethanol. 87% yield.

³¹P NMR (161.97 MHz, CDCl₃): δ 37.46. ¹H NMR (400.13 MHz, CDCl₃): δ 1.38 (s, 3H, CH₃), 1.41 (s, 3H, CH₃), 1.44 (s, 3H, CH₃), 1.50 (s, 3H, CH₃), 1.86 (bs, 2H, NH₂), 3.49 (dd, 1H, ²J_{P-H}=5.2 Hz, ³J_{H-H}=6.4 Hz, ¹CH), 4.11 (m, 2H, ⁶CH₂), 4.39-4.58 (m, 4H, ³CH+⁵CH+²CH+⁴CH), 7.39-7.87 (m, 5H, CH_{Ar}). ¹³C NMR (100.61 MHz, CDCl₃): δ 25.04 (s, 1C, CH₃), 25.27 (s, 1C, CH₃), 26.93 (s, 1C, CH₃), 26.95 (s, 1C, CH₃), 48.78 (d, 1C, ¹J_{P-C}=91.6 Hz, ¹CH), 66.78 (s, 1C, ⁶CH₂), 72.85 (d, 1C, ²J_{P-C}=3 Hz, ²CH), 73.27 (d, 1C, ²J_{P-C}=5.1 Hz, ⁴CH), 73.97 (d, 1C, ³J_{P-C}=9.5 Hz, ³CH), 78.33 (s, 1C, ⁵CH), 109.23 (d, 1C, Cq), 109.96 (d, 1C, Cq), 128.71 (d, 1C, ²J_{P-C}=138 Hz, Cq_{Ar}), 128.41 (s, 1C, CH_{Ar}), 128.52 (s, 1C, CH_{Ar}), 131.92 (s, 1C, CH_{Ar}), 132.02 (s, 1C, CH_{Ar}), 133.02 (s, 1C, CH_{Ar}P). IR (KBr pellet): 3287 f (v OH), 2987 ff, 2937 if (v CH_{Alk}), 1593 ff (v NH), 1439 ff, 1372 f, 1207 FF (v P═O), 1159 m (v C—O—C), 1124 F (v C—O), 1060 FF, 958 F (v P—O—C), 920 f, 890 m, 836 m, 798 f, 727 F, 694 F. MS FAB+ (NBA): m/z (%) 767 [2M+H⁺] (10%), 384 [M+H⁺] (90%). HR MS⁺ (NBA): Theoretical mass 384.1576; Experimental mass 384.1567.

### Synthesis of compound 5.6d (R_{P}SSRR)-3-Acetamide-4-2.2-dimethyl-[1.3]dioxolan-4-yl)-2.2-dimethyl-2-oxo-2-phenyltetrahydro-2λ⁵-[1.2]oxaphosphinane

Acetic anhydride (72 μl, 0.76 mmol) and triethylamine (60 μl, 0.42 mmol) are added to a solution of compound 5.5d obtained previously (145 mg, 0.38 mmol) in anhydrous CH₂Cl₂ (5 ml), under a nitrogen atmosphere. The reaction mixture is stirred at room temperature for 2 hours. The organic solution is washed with saturated aqueous sodium hydrogen carbonate solution. The combined organic phases are dried over sodium sulfate, filtered and concentrated under vacuum. The residue is then purified by chromatography on silica gel (50/50/0 to 0/95/5 heptane/EtOAc/EtOH gradient). The colorless solid obtained is dissolved in the minimum necessary amount of diethyl ether and reprecipitated from hexane. Compound 5.6d is obtained in an overall yield of 12% (20 mg).

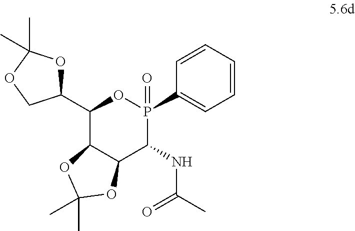 [SMILES: CC(=O)N[C@@H]1[C@H]2OC(C)(C)O[C@H]2[C@@H]([C@H]2COC(C)(C)O2)O[P@@]1(=O)c1ccccc1]

³¹ NMR (161.97 MHz, CDCl₃): δ=37.02 ppm. ¹H NMR (400.13 MHz, CDCl₃): δ=1.31 (s, 3H), 1.34 (s, 3H), 1.37 (s, 3H), 1.53 (s, 3H), 1.91 (s, 3H), 4.03 (ddd, 2H, J=4.5, 5.5, 9 Hz), 4.36-4.48 (m, 3H), 4.60-4.71 (m, 2H,), 6.74 (bs, 1H), 7.42-7.88 (m, 5H) ppm. ¹³C NMR (100.61 MHz, CDCl₃): δ=25.59, 26.41, 26.85, 27.51, 66.61, 68.01, 68.97, 74.72, 75.09, 75.17, 76.86, 76.93, 109.88, 111.04, 129.70, 130.03, 130.54, 131.92, 133.87, 135.50, 165.98. HRMS (ESI) m/z theoretical for C₂₀H₃₀NO₇P (M+H)⁺: 426.1682; Experimental mass 426.1686.

### c) Formulation of the Compounds of Formula (1) According to the Invention

The compounds of formulae 3.48a and 4.2a are insoluble in water, in numerous water-soluble solvents, and non-complexible in cyclodextrins. The following two formulations are particularly suited to compounds 3.48a and 4.2a and allow their in vivo administration, at concentrations of about 20-100 mg/kg, intravenously:

### Formulation 1:

Dissolution of compounds 3.48a and 4.2a in an EtOH/MDA mixture followed by extemporaneous addition of surfactant Solutol® HS15 (polyethylene glycol-660-hydroxystearate sold by the company BASF) heated to 37° C. and filtered (0.22 μm) to obtain an EtOH/MDA/Solutol® HS15 mixture (1/3/6 by volume) diluted by adding 10% by volume of a 0.9% NaCl solution). The 0.9% NaCl solution used is an injectable solution (sterile and apyrogenic), in sterile glass bottles with hermetic sealing.

### Formulation 2:

Prepared in the same manner as formulation 1, to give an EtOH/MDA/Solutol® HS15 mixture (1/3/6 by volume), diluted fourfold with 0.9% NaCl (except during the preparation of a formulation of compound 3.48a or 4.2a at 5 mg/kg).

### d) Acute and Chronic In Vivo Toxicity

Studies of the acute toxicity of compounds 3.48a and 4.2a were performed on healthy mice for a concentration range of 50, 25, 10, 5 and 1 mg/kg. No mortality was observed, even for the maximum dose of 50 mg/kg.

A chronic toxicity study was performed for injections of 20, 5 and 1 mg/kg at a rate of three injections/week for four weeks. No mortality or weight loss was observed, even for the maximum dose.

### e) In Vivo Antimetastatic Activity

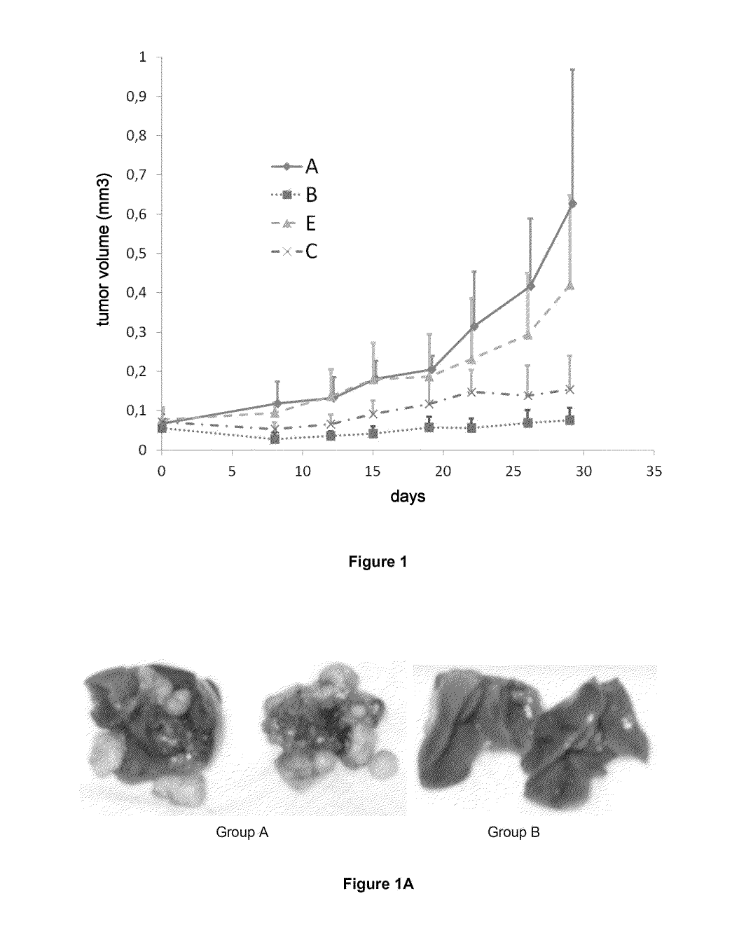

SNB75 Subcutaneous Model (FIG. 1)

A suspension of 5×10⁶ SNB75 cells in 100 μl of sterile PBS (phosphate-buffered saline, provided by Fisher) was injected into each mouse (subcutaneous administration without anesthesia). Once the mice presented tumors with volumes of between 0.13 and 0.033 mm³, groups were formed so that each of them contained an equivalent number of large, medium and small tumors. The protocol was conducted with five groups of seven mice. The five groups of mice were treated using one of the five formulations described below:

Group A: Treatment by injection of 100 μl of NaCl (control N° 1).

Group B: Treatment with compound 3.48a at a rate of 10 mg/kg with volumes of 100 to 150 μl of formulation 2 according to the weight of the mice.

Group C: Treatment with compound 3.48a at a rate of 5 mg/kg with volumes of 100 to 150 μl of formulation 2 according to the weight of the mice.

Group D: Treatment with temozolomide at a rate of 20 mg/kg with volumes of 100 to 150 μl according to the weight of the mice. Temozolomide is a comparative anticancer active principle sold under the name Temodal by the laboratory Schering Plough. It is indicated for treating glioblastoma multiforme. The dose was limited to 20 mg/kg since a dose of 40 mg/kg leads to the death of the mice before the end of the experimentation period.

Group E: Treatment by injection of 100 to 150 μl according to the weight of the mice of formulation 2 not containing compound 3.48a (control N° 2).

Each batch was treated by intravenous injection into the caudal vein of 100 to 150 μl of solution (precision syringe and G25X1 needle ultrafine wall, VWR). Three injections per week for four weeks were performed. The mice were weighed at each injection. The sizes of the tumors were measured twice a week for a period of four weeks. The mice were sacrificed after four weeks and the tumors and organs (liver, kidney, spleen, brain, intestine, heart, muscles) were removed. A histological analysis was performed on these organs.

As shown by FIG. 1, a very great reduction in the growth of the primary tumor was observed in the mice treated with compound 3.48a (groups B and C) after four weeks, compared with the mice of the control groups (groups A and E). For groups B and C, none of the mice died and no weight loss was observed. As regards batch D, three of the seven mice died after the third injection, and substantial weight losses were observed for the remaining four mice, despite a very large regression of the tumor.

The SNB75 cell line originating from a human glioblastoma is capable of metastasizing in nude mice following subcutaneous implantation. Specifically, all the mice of the control groups (groups A and E) developed metastases in the liver and the intestine. None of the mice treated with compound 3.48a (groups B and C) developed metastases. FIG. 1A shows the presence of metastases in the liver of a mouse from group A and the absence of metastases in the liver of a mouse from group B, eight weeks after subcutaneous implantation of the SNB75 cells.

Gli4 Intracranial Model

2×10⁵ Gli4 cells were injected into a group of mice (right striatal injection, injection coordinates: bregma 0 L-L: L: −1.5 mm P: −3.5-2.5). The mice were immediately treated for four weeks at a rate of three injections at 10 mg/kg of compound 3.48a per week (formulation 1, group B) or using 100 μl of NaCl according to the same treatment protocol (group A) and sacrificed after ten weeks.

In the group of control mice (group A), dispersed Gli4 cells are observed, which migrated along the corpus callosum and passed into the other hemisphere. The Gli4 cells were identified with an anti-human nucleus (anti-HuNu) antibody revealed with an anti-mouse secondary antibody (Alexa 594). This migration reflects migration along the white matter, as observed in human glioblastoma multiformes (dispersion as an intra-fascicular growth along the white matter).

In the group of mice treated with compound 3.48a at a rate of 10 mg/kg (group B), the four-week treatment made it possible to inhibit cell migration. After stopping the treatment, resumption of the growth and migration of the cells was observed in the final six weeks.

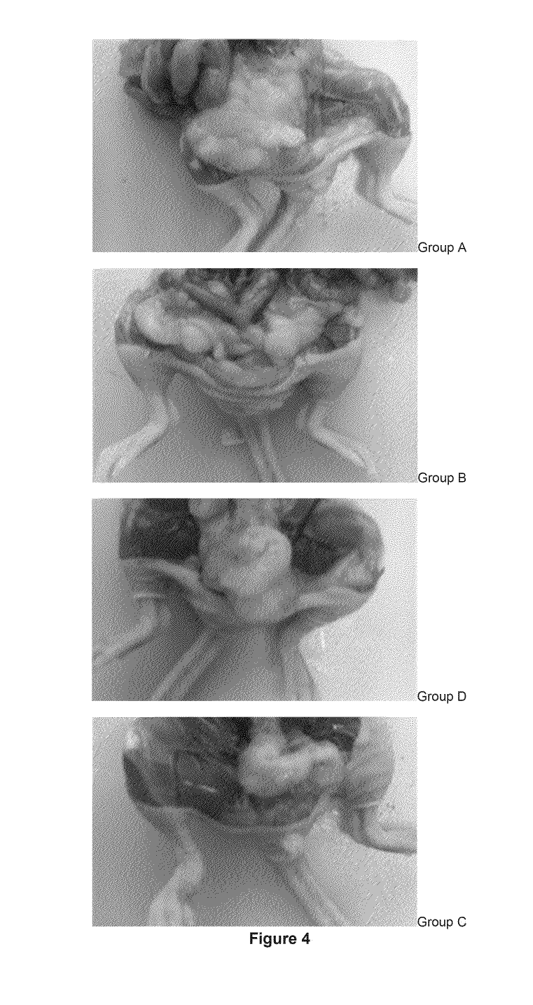

MDAMB-435 Subcutaneous Model (FIGS. 4-6)

A suspension of 1×10⁶ MDAMB-435 cells in 100 μl of sterile PBS (phosphate-buffered saline, supplied by Fisher) was injected into each mouse (subcutaneous administration, with anesthesia, in the mammary gland of the left hind quarter). Once a control pregroup of three mice presented the first metastases (liver, intestine, ovary), the protocol was conducted with four groups of eight mice. The five groups of mice were treated using one of the five formulations described below:

Group A: Treatment by injection of 100 μl of NaCl (control N° 1).

Group B: Treatment by injection of 100 to 150 μl according to the weight of the mice of formulation 2 not containing compound 4.2a (control N° 2).

Group C: Treatment with compound 4.2a at a rate of 10 mg/kg with volumes of 100 to 150 μl of formulation 2 depending on the weight of the mice.

Group D: Treatment with compound 4.2a at a rate of 5 mg/kg with volumes of 100 to 150 μl of formulation 2 depending on the weight of the mice.

Each batch was treated by intravenous injection into the caudal vein of 100 to 150 μl of solution (precision syringe and G25X1 needle ultrafine wall, VWR). Three injections per week for four weeks were performed. The mice were weighed at each injection. The mice were sacrificed after four weeks, and the tumors and organs (liver, kidney, spleen, brain, intestine, heart, muscles) were removed. A histological analysis was performed on these organs.

The MDAMB-435 cell line originating from a human melanoma is capable of metastasizing in nude mice after subcutaneous implantation into the mammary glands. Specifically, all the mice of the control groups (groups A and B) developed metastases in the liver and the intestine. The mice treated with compound 4.2a (groups C and D) underwent a reduction in metastatic development (small-sized metastases) and a large reduction in the number of metastases.

FIG. 4 shows the presence of metastases in the ovaries in the control mice (groups A and B), and the absence of metastases on the mice treated with compound 4.2a (groups C and D).

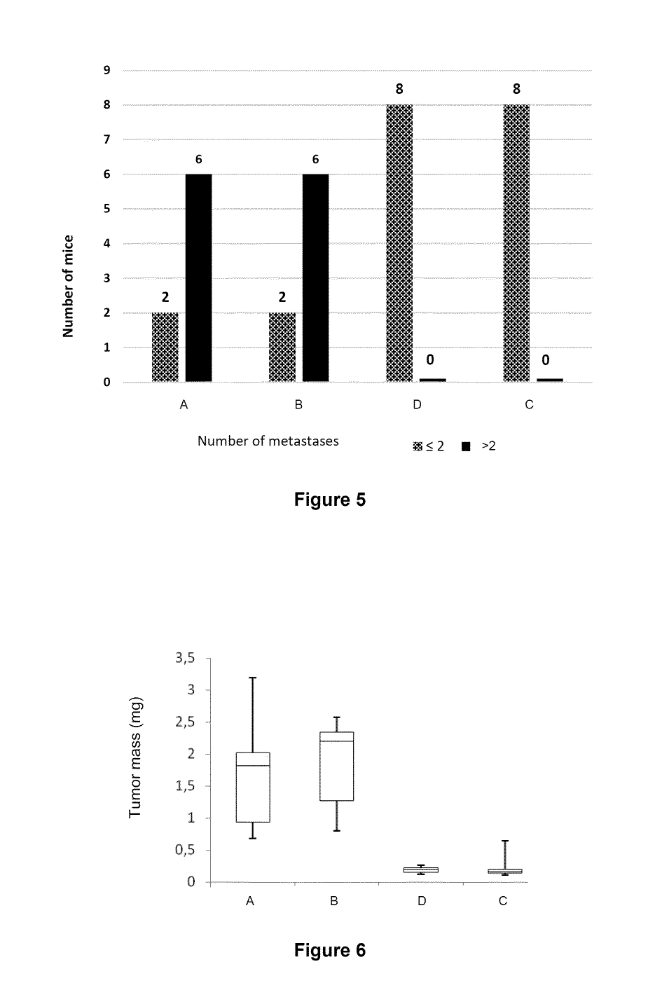

FIG. 5 shows the mean number of metastases per mouse as a function of the treatment conditions. It shows that none of the mice treated with compound 4.2a (groups C and D) developed more than two metastases. On the other hand, the control mice (groups A and B) predominantly developed more than two metastases.

FIG. 6 shows the mass distribution of these secondary tumors as a function of the treatment conditions. It shows that treatment with compound 4.2a reduced the development of metastases.

### f) In Vitro Antiproliferative Activity

The lines MDAMB-435 and A431 are seeded at a density of 3000 cells per well in 96-well plates. After 24 hours, compounds 4.2a and 5.6d resuspended in 100% DMSO are added at concentrations of 0-0.001-0.010-0.1-1-10-50 μM for a final constant amount of DMSO of 0.3%. The cultures are maintained for a further 72 hours and an MTT colorimetric test is then performed. 100 μl of a solution of MTT at 5 mg/ml in PBS are added to the culture medium, which is left to incubate for 3 hours. The culture medium is removed and the cells are lyzed by adding 100 μL of a 4 mM HCl solvent, 0.1% Nondet P-40 (NP40) in isopropanol. The yellow tetrazolium MTT salt (3-(4,5-dimethylthiazol-2-yl)-2,5-diphenyl tetrazolium bromide), when it is reduced by the mitochondrial succinate dehydrogenase of active live cells, forms a blue-violet formazan product. The medium then changes from yellow to blue-violet. The intensity of this coloration at 590 nm is proportional to the number of live cells present during the test and gives an indication of the metabolic activity of these cells.

Table 1 gives the concentration values for 50% inhibition of proliferation (IC₅₀) for compounds 4.2a and 5.6d on lines derived from bowel cancer (CaCo2), skin cancer (A431, MDAMB-435, B16F10), breast cancer (MDAMB-431), intestinal cancer (HuH7), prostate cancer (DU145) and brain cancer (SNB75, Gli4, Gli7).

TABLE 1
Antiproliferative effect of compounds 4.2a and 5.6d on various cancer cell lines

| | | Cell lines | | | | | | | | | | |
|---|---|---|---|---|---|---|---|---|---|---|---|---|
| | | | | MDAMB- | MDAMB- | | | | | | | |
| | Compounds | A431 | CaCo2 | 231 | 435 | HuH7 | B16F10 | DU145 | C6 | SNB75 | GLi4 | Gli7 |
| IC₅₀ | 4.2a | 4.35 ± | >100 | 1.16 ± | 0.66 ± | >100 | 13.48 ± | >100 | 8.51 ± | 9.54 ± | 6.81 ± | 2.71 ± |
| μM | | 0.40 | | 0.00 | 0.00 | | 0.03 | | 0.00 | 0.00 | 0.00 | 0.00 |
| | 5.6d | 0.13 ± | 35.96 ± | 23.17 ± | 9.61 ± | 21.95 ± | 14.87 ± | 31.16 ± | 0.65 ± | 0.65 ± | 0.54 ± | 0.50 ± |
| | | 0.00 | 0.02 | 0.01 | 0.03 | 0.00 | 0.01 | 0.01 | 0.00 | 0.00 | 0.00 | 0.00 |

Table 1 shows that compounds 4.2a and 5.6d have antiproliferative activity on various cancer lines of different human and rat tissues. In particular, compound 4.2a inhibits the cell proliferation of the human melanoma line MDAMB-435 with an IC₅₀ of 0.66 μM and compound 5.6d inhibits the proliferation of the A431 line (human epidermoid cancer) with an IC₅₀ of 100 nM.

### g) In Vitro Antimigratory Activity

The effects of compounds 3.48a, 4.2a and 5.6d on the migration of cancer cells (human glioblastoma line SNB75 or primary culture of human glioblastoma Gli4F1, on the one hand, and MDAMB 435 and A431 on the other hand), were determined by performing a standard migration test in a Boyden chamber (treatment of the cells for 24 hours).

For this test, the compounds were dissolved in vitro and used in culture in 0.3% of DMSO. The interior of the inserts was covered with the extracellular matrix protein studied (laminin-1, vitronectin or fibronectin). The cells pretreated at various concentrations and untreated (controls) were deposited in the inserts covered with said protein at a rate of 50 000 cells/500 μl/insert in a medium containing 0.1% of BSA (bovine serum albumin).

500 μl of medium containing 10% fetal calf serum were then placed in each well of a Companion plate so as to allow the formation of a chemotactic gradient across the porous membrane. After 24 hours of migration in the incubator at 37° C., the supernatent was removed and the inserts rinsed with PBS on each face. The cells were counted with a microscope (Zeiss Axiophot, magnification ×200). This gives a percentage of migration relative to the untreated cells.

Table 2 below gives, for compound 3.48a, the values for the inhibition constants (Ki) for the migration of a human GBM line (SNB75), of GBM stem cell lines (Gli4, Gli7) and of a breast cancer line MDAMB231, in a Boyden chamber, on various extracellular matrix protein supports. Tables 3 and 4 below give, for compounds 4.2a and 5.6d, the values of the inhibition constants (Ki) for the migration of a melanoma line (MDAMB-435) or of an epidermoid cancer line (A431), in a Boyden chamber, on various extracellular matrix protein supports. The inhibitory effect depends on the line and the stem cells used, but also on the extracellular matrix protein support used.

TABLE 2
Antimigratory effect of compound 3.48a on various
lines and stem cells of glioblastoma multiforme

| | | Support | | |
|---|---|---|---|---|
| | | Fibronectin | Laminin | Vitronectin |
| | | Inhibition constant Ki (nmol) | | |
| Lines | SNB75 | 27 | 23 000 | 87 |
| | Gli4 | 1 | 11 | 5 |
| | Gil7 | 37 | 310 | 371 |

TABLE 3
Antimigratory effect of compound 4.2a
on the melanoma line MDAMB-435

| | Support | | |
|---|---|---|---|
| | Fibronectin | Laminin | Vitronectin |
| | Inhibition constant Ki (nmol) | | |
| MDAMB-435 line | 33 | 34 | 61 |

TABLE 4
Antimigratory effect of compound 5.6d
on the epidermoid cancer line A431

| | Support | | |
|---|---|---|---|
| | Fibronectin | Laminin | Vitronectin |
| | Inhibition constant Ki (nmol) | | |
| A431 line | 8 | 13 | 20 |

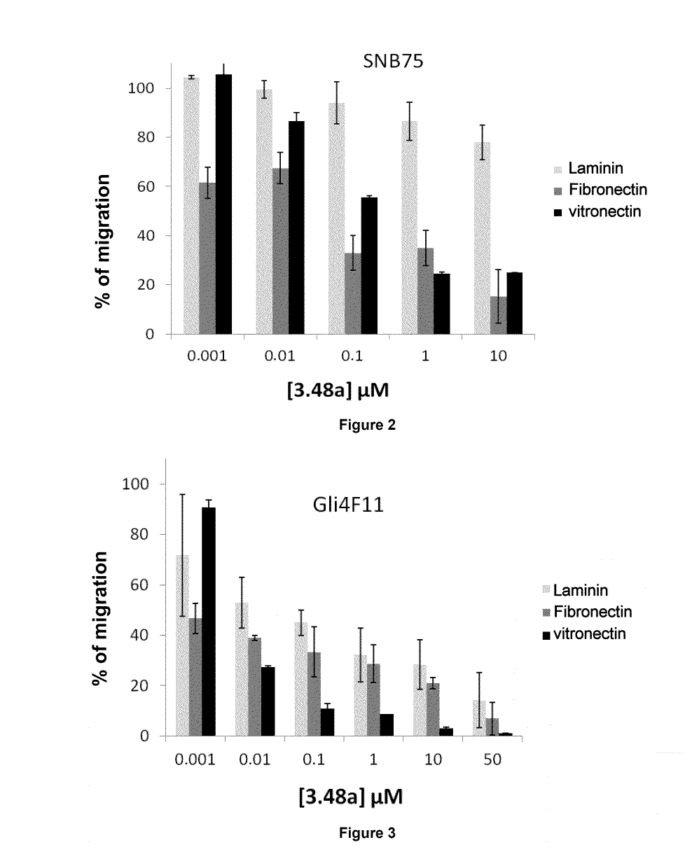

FIGS. 2 and 3 and Table 2 shows that compound 3.48a inhibits the cell migration of the GBM lines SNB75 and C6 and GBM stem cells (Gli4 and Gli7) on various extracellular matrix substrates with a dose effect that is dependent on the substrate used (vitronectin, fibronectin or laminin-1). A 50% decrease in migration of the cancer cells is specifically obtained on fibronectin or vitronectin for a concentration of compound 3.48a as low as 1 to 100 nM (SNB75 line).

Tables 3 and 4 show that compounds 4.2a and 5.6d inhibit, respectively, the cell migration of the lines MDAMB-435 and A431 with a dose effect that is dependent on the substrate used (vitronectin, fibronectin or laminin-1). A 50% decrease in migration of the cancer cells is especially obtained on fibronectin or vitronectin for a concentration of compound 4.2a and 5.6d as low as 10 to 100 nM.

The compounds (1) according to the invention have a mode of action different from the mode of action of the compounds on the market that are assumed to be antimetastatic. Without wishing to be bound by a theory, the inventors think that the compounds (1) target cellular glycosylation by inhibiting the glycosyltransferases that catalyze the addition of N-acetylglucosamines in the beta-1,6 position on the alpha-1,6-bonded mannose groups. The consequences of these modifications are an inhibition of the migratory potential of these cells on various extracellular matrix supports such as fibronectin or vitronectin.

1. A compound for a use for the purpose of reducing or preventing the appearance of metastases in a patient afflicted with a cancer, corresponding to the general formula below: 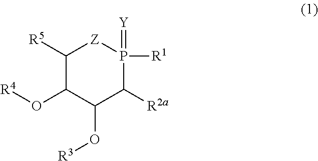
in which:
Y represents an oxygen, sulfur or selenium atom, preferably an oxygen atom,
Z represents O, S, Se, NH or a group NR⁶, in which R⁶ is an optionally substituted aryl or alkyl group, preferably an oxygen atom,
R¹ represents a hydrogen atom, an optionally substituted alkyl or cycloalkyl group, or an aryl or heteroaryl group,
R^{2a} represents a hydrogen or halogen atom, an azide group (N₃), a carbonate or dithiocarbonate group, a 1H-[1,2,3]triazolyl group or a group —X—R², X representing an oxygen, sulfur or selenium atom, a group NH or NR⁷, in which R⁷ is an optionally substituted aryl, heteroaryl, alkyl or cycloalkyl group, X preferably representing O or NH, and R² representing an optionally substituted aryl, heteroaryl, alkyl or cycloalkyl group, a hydrogen atom, a trichloroacetimidate group (—C(═NH)CCl₃), acyl, formyl, sulfonyl, sulfinyl, tert-butyldiphenylsilyl, allyl, a saccharyl, ester, amide, thioamide or sulfonamide group, or X—R² represents a group P(O)R^{2′}R^{6′} in which R^{2′} and R^{6′} denote, independently of each other, an optionally substituted aryl, heteroaryl, alkyl or cycloalkyl group, OH, alkoxy or aryloxy,
R³ and R⁴ represent, independently of each other, an optionally substituted aryl, heteroaryl, alkyl or cycloalkyl group, a hydrogen atom, a trichloroacetimidate, acyl, formyl, sulfonyl, sulfinyl, tert-butyldiphenylsilyl, allyl, ester, amide, thioamide, sulfonamide or saccharyl group or alternatively R³ and R⁴, taken together, form a divalent radical of formula —R³—R⁴—, in which —R³—R⁴— preferably represents an isopropylidene, benzylidene, diphenylmethylidene, cyclohexylmethylidene group, optionally substituted, preferably a 4-methoxybenzylidene group, or a linear alkylene group, preferably the ethylene group,
R⁵ represents a hydrogen atom or a hydrocarbon-based group comprising one or more heteroatoms preferably chosen from oxygen, sulfur and nitrogen, better still oxygen.

2. The compound as claimed in claim 1, wherein Y=Z=O.

3. The compound as claimed in claim 1, wherein the group R⁵ is chosen from the groups: 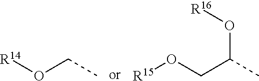
in which R¹⁴, R¹⁵ and R¹⁶ represent, independently of each other, a hydrogen atom, an optionally substituted aryl, heteroaryl, alkyl or cycloalkyl group, a trichloroacetimidate, acyl, formyl, sulfonyl, sulfinyl, tert-butyldiphenylsilyl, allyl, ester, amide, sulfonamide or saccharyl group or alternatively R¹⁵ and R¹⁶, taken together, form a divalent radical of formula —R¹⁵—R¹⁶—, in which —R¹⁵—R¹⁶— preferably represents an isopropylidene, benzylidene, diphenylmethylidene or cyclohexylmethylidene group, which are optionally substituted, for example a 4-methoxybenzylidene group, or a linear alkylene group such as the ethylene group.

4. The compound as claimed in claim 1, wherein the compound is chosen from the compounds of formula (2) or (3): 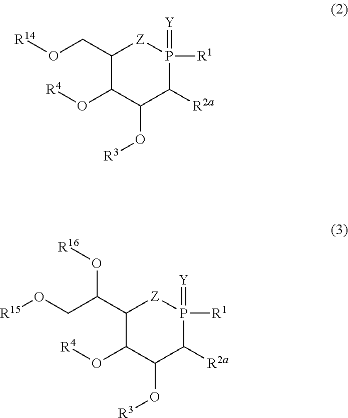
in which R¹, R^{2a}, R³, R⁴, Y and Z are as previously defined, R¹⁴, R¹⁵ and R¹⁶ represent, independently of each other, a hydrogen atom, an aryl or heteroaryl group, an optionally substituted alkyl or cycloalkyl group, a trichloroacetimidate group, an acyl, formyl, sulfonyl, sulfinyl, tert-butyldiphenylsilyl, allyl, ester, amide, thioamide, sulfonamide or saccharyl group or alternatively R¹⁵ and R¹⁶, taken together, form a divalent radical of formula —R¹⁵—R¹⁶—.

5. The compound as claimed in claim 1, wherein the compound is chosen from the compounds of formula: 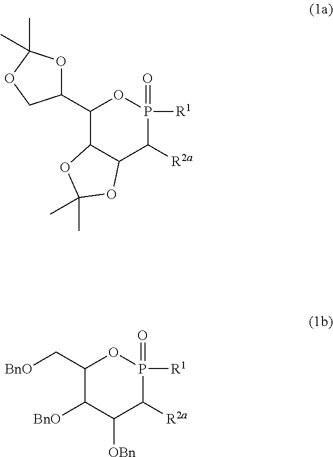
in which R¹ and R^{2a} are as previously defined, and Bn represents the benzyl group.

6. The compound as claimed in claim 1, wherein the compound is chosen from the compounds of formula: 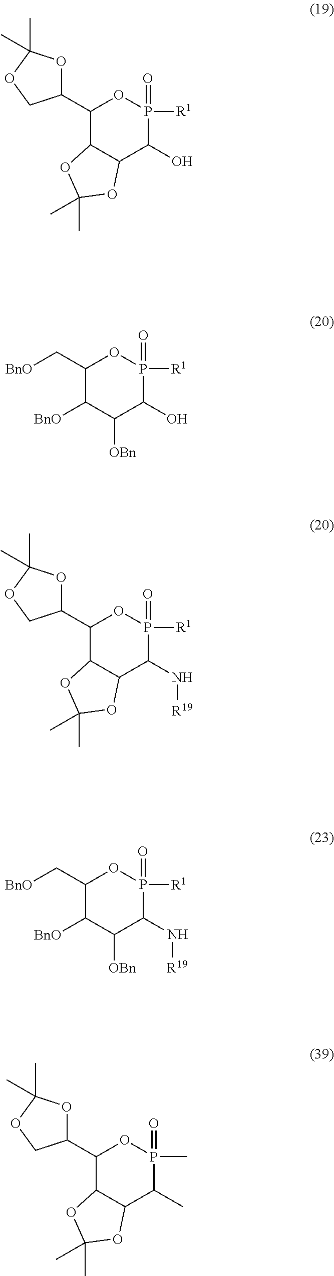
in which R¹ is as previously defined, Bn represents the benzyl group, R¹⁹ and R³² represent, independently of each other, a hydrogen atom, an aryl or heteroaryl group, an optionally substituted alkyl or cycloalkyl group, an acyl group.

7. The compound as claimed in claim 1, wherein R^{2a} represents a group —X—R², in which X=NH.

8. The compound as claimed in claim 7, wherein X—R² is NHC(O)R¹², in which R¹² represents an aryl or heteroaryl group, an optionally substituted alkyl or cycloalkyl group.

9. The compound as claimed in claim 1, wherein R^{2a} represents a group —X—R², in which R² is an aryl or heteroaryl group, preferably a heteroaryl group.

10. A pharmaceutical composition for a use for the purpose of reducing or preventing the appearance of metastases in a patient afflicted with a cancer, which comprises at least one compound of formula (1) as defined in claim 1, in combination with one or more pharmaceutically acceptable excipients and/or vehicles.

11. The pharmaceutical composition according to claim 10, wherein the patient is afflicted with a cancer chosen from glioblastoma multiforme, breast cancer and non-small-cell lung cancer, preferably glioblastoma multiforme.

12. The pharmaceutical composition as claimed in claim 10, which comprises at least one organic solvent and at least one surfactant.

13. A combination product for a use in the treatment of a cancer, which comprises:
i) At least one cytotoxic compound, and
ii) At least one compound of formula (1) as defined in claim 1, for a use for the purpose of reducing or preventing the appearance of metastases.

14. The compound as claimed in claim 2, wherein the group R⁵ is chosen from the groups:

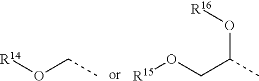

15. The pharmaceutical composition as claimed in claim 11, which comprises at least one organic solvent and at least one surfactant.
